SEQUENCE LISTING (26 sequences)

SEQ ID NO: 1 - protein, 379 aa, Homo sapiens
MEQLSSANTRFALDLFLALSENNPAGNIFISPFSISSAMAMVFLGTRGNTAAQLSKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFCMLMPEENFTADHPFLFFIRHNSSGSILFLGRFSSP

SEQ ID NO: 2 - protein, 379 aa, Artificial Sequence
  Description of Artificial Sequence Synthetic polypeptide
MEQLSSANTRFALDLFLALSENNPAGNIFISPFSISSAMAMVFLGTRGNTAAQLSKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFGMLMPEENFTADHPFLFFIRHNSSGSILFLGRFSSP

SEQ ID NO: 3 - protein, 379 aa, Artificial Sequence
  Description of Artificial Sequence Synthetic polypeptide
MEQLSSANTRFALDLFLALSENNPAGNIFISPFSISSAMAMVFLGTRGNTAAQLSKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFAMLMPEENFTADHPFLFFIRHNSSGSILFLGRFSSP

SEQ ID NO: 4 - protein, 379 aa, Artificial Sequence
  Description of Artificial Sequence Synthetic polypeptide
MEQLSSANTRFALDLFLALSENNPAGNIFISPFSISSAMAMVFLGTRGNTAAQLSKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFVMLMPEENFTADHPFLFFIRHNSSGSILFLGRFSSP

SEQ ID NO: 5 - protein, 379 aa, Artificial Sequence
  Description of Artificial Sequence Synthetic polypeptide
MEQLSSANTRFALDLFLALSENNPAGNIFISPFSISSAMAMVFLGTRGNTAAQLSKTFHFNTVEEVHSRFQSLNADINKRGASYILKLANRLYGEKTYNFLPEFLVSTQKTYGADLASVDFQHASEDARKTINQWVKGQTEGKIPELLASGMVDNMTKLVLVNAIYFKGNWKDKFMKEATTNAPFRLNKKDRKTVKMMYQKKKFAYGYIEDLKCRVLELPYQGEELSMVILLPDDIEDESTGLKKIEEQLTLEKLHEWTKPENLDFIEVNVSLPRFKLEESYTLNSDLARLGVQDLFNSSKADLSGMSGARDIFISKIVHKSFVEVNEEGTEAAAATAGIATFSMLMPEENFTADHPFLFFIRHNSSGSILFLGRFSSP

SEQ ID NO: 6 - DNA, 1000 bp, Saccharomyces cerevisiae
tataccaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaat

SEQ ID NO: 7 - protein, 4 aa, Artificial Sequence
  Description of Artificial Sequence Synthetic peptide
  N-term Succ
  C-term para-nitroanilide
AAPV

SEQ ID NO: 8 - protein, 4 aa, Artificial Sequence
  Description of Artificial Sequence Synthetic peptide
  N-term Succ
  C-term para-nitroanilide
AAPF

SEQ ID NO: 9 - protein, 15 aa, Homo sapiens
ATAGIATFCMLMPEE

SEQ ID NO: 10 - protein, 6 aa, Artificial Sequence
  Description of Artificial Sequence Synthetic peptide
GIATFM

SEQ ID NO: 11 - protein, 6 aa, Artificial Sequence
  Description of Artificial Sequence Synthetic peptide
TFMLMP

SEQ ID NO: 12 - protein, 6 aa, Artificial Sequence
  Description of Artificial Sequence Synthetic peptide
FMLMPE

SEQ ID NO: 13 - protein, 15 aa, Homo sapiens
TEAAAATAGIATFCM

SEQ ID NO: 14 - protein, 15 aa, Homo sapiens
TEAAAATAAIMMMRC

SEQ ID NO: 15 - protein, 15 aa, Homo sapiens
TEAAAATAVVRNSRC

SEQ ID NO: 16 - protein, 16 aa, Homo sapiens
TEAAAASSCFVVAECC

SEQ ID NO: 17 - protein, 21 aa, Homo sapiens
EEGTEAAAATAGIATFCMLMP

SEQ ID NO: 18 - protein, 21 aa, Mus musculus
EEGTEAAAATGGIATFCMLLP

SEQ ID NO: 19 - protein, 21 aa, Sus scrofa
EEGTEAAAATAGIAVFAMLMP

SEQ ID NO: 20 - protein, 21 aa, Equus caballus
EEGTEAAAATAGTIMLAMLMP

SEQ ID NO: 21 - protein, 21 aa, Rattus norvegicus
EEGTEAAAATAGIATFCMLLP

SEQ ID NO: 22 - protein, 21 aa, Macaca fascicularis
EEGTEAAAATAGVATFCMLLP

SEQ ID NO: 23 - protein, 21 aa, Danio rerio
EEGTEAAAATAAIVAFCMLRE

SEQ ID NO: 24 - protein, 21 aa, Bos Taurus
EEGTEAAAATGAIAEFAMLVP

SEQ ID NO: 25 - protein, 21 aa, Ovis aries
EEGTEAAAATGAIAQFAMLMP

SEQ ID NO: 26 - protein, 21 aa, Xenopus laevis
EEGTEAAAASAGIAMMCMMRE

(19) United States
(12) Patent Application Publication
(10) Pub. No.: US 2022/0267412 A1
(43) Pub. Date: Aug. 25, 2022

(54) OXIDATION-RESISTANT SERPINS

(71) Applicant: SERPLUS TECHNOLOGY LLC, Belmont, CA (US)

(72) Inventor: Philip A. PEMBERTON, Belmont, CA (US)

(21) Appl. No.: 17/631,800

(22) PCT Filed: Jul. 31, 2020
(86) PCT No.: PCT/US2020/044604

§ 371 Date: Jan. 31, 2022

Related U.S. Application Data
(60) Provisional application No. 62/881,858, filed on Aug. 1, 2019.

(51) Int. Cl. C07K 14/81; A61K 38/55; A61K 9/00; A61P 9/00; A61P 35/00; A61P 11/00; C12N 15/10

(52) U.S. Cl. CPC C07K 14/8121; A61K 38/55; A61K 9/0073; A61K 9/0014; A61K 9/0019; A61P 9/00; A61P 35/00; A61P 11/00; C12N 15/102; C12N 2330/50; C07K 2317/622; C07K 2317/52

(57) ABSTRACT

This disclosure provides SERPIN B1 polypeptides that possess neutrophil or pancreatic elastase inhibitory activity, and the elastase inhibition activity is resistant to oxidation by free radicals. The free radicals may a reactive oxygen species, or a reactive nitrogen species, or both. In some embodiments, the SERPIN B1 polypeptide comprises an amino acid substitution at residue 344 as compared to SEQ ID NO: 1. The SERPIN B1 polypeptides disclosed herein can be used to treat a patient having a disease or a genetic condition that is associated with the increased production of free radicals as compared to a normal individual or increased exposure to free radicals in environmental sources.

### REFERENCE TO RELATED APPLICATION

The application claims the benefit of, and priority to, U.S. Provisional Application No. 62/881,858, filed on Aug. 1, 2019. The entire content of said provisional application is herein incorporated by reference for all purposes.

### BACKGROUND

Human serpin B1 is a member of the serine proteinase inhibitor (serpin) superfamily that has anti-inflammatory properties. The anti-inflammatory properties are, in part, attributable to its ability to inhibit pro-inflammatory neutrophil serine proteases (NSP's) via the reactive site loop (RSL) and the ability to limit the self-association and spontaneous activation of pro-caspases via a caspase recruitment domain binding motif (CBM) located C-terminal to the RSL (Cooley, et al., 2001; Choi et al., 2019) Human serpin B1 can inhibit the NSP's cathepsin G and elastase through efficient reactions at 2 overlapping reactive sites: Phe-343 and Cys-344.

### SUMMARY

In some aspects, this disclosure provides a SERPIN B1 variant polypeptide that comprises an amino acid sequence that is at least 90% identical to SEQ ID NO: 1, and the SERPIN B1 variant polypeptide possesses neutrophil or pancreatic elastase inhibitory activity and the elastate inhibition activity of the SERPIN B1 variant polypeptide is resistant to oxidation by free radicals. The free radicals may be reactive oxygen species, or reactive nitrogen species, or both. In some embodiments, the SERPIN B1 variant polypeptide comprises an amino acid substitution at residue 344 as compared to SEQ ID NO: 1. In some embodiments, the amino acid substitution is selected from the group consisting of C344A, C344V, and C344G.

In some embodiments, the SERPIN B1 variant polypeptide disclosed herein is fused to an Fc portion of an IgG, a single chain variable fragment (scFv) of an antibody. In some embodiments, the SERPIN B1 variant polypeptide is pegylated.

Also provided herein is a polynucleotide encoding any of the SERPIN B1 variant polypeptides disclosed in this disclosure.

Also provided herein is a pharmaceutical composition comprising any of the SERPIN B1 polypeptides disclosed in this application and a pharmaceutically acceptable excipient. In some embodiments, a pharmaceutical composition comprises a SERPIN B1 polypeptide comprising an amino acid sequence that is at least 90% identical to SEQ ID NO: 1 and a reducing agent that prevents oxidation of cysteine 344, wherein the polypeptide is capable of inhibiting neutrophil or pancreatic elastase. In some embodiments, the reducing agent is N-acetylcysteine (NAC).

Also provided herein is a method of treating a patient having a disease that is associated with increased production of free radicals as compared to a normal individual or increased exposure to free radicals in environmental sources. The method comprises administering a SERPIN B1 variant polypeptide, wherein the SERPIN B1 variant polypeptide comprises an amino acid sequence that is at least 90% identical to SEQ ID NO:1, wherein the SERPIN variant polypeptide comprises an amino acid substitution at residue 344, as compared to the native protein sequence of SEQ ID NO: 1; said SERPIN B1 variant polypeptide is capable of inhibiting the serine protease activity of a neutrophil or pancreatic elastase and is resistant to oxidation by a free radical. In some embodiments, the free radical is a reactive oxygen species, a reactive nitrogen species, or both. In some embodiments, the SERPIN variant polypeptide comprise an amino acid sequence selected from the group consisting of SEQ ID NO: 2-4.

Also provided herein is a method of treating a patient having a disease or a genetic condition that is associated with the increased production of free radicals as compared to a normal individual or increased exposure to free radicals in environmental sources, wherein the method comprises administering the pharmaceutical composition of comprising the SERPIN B1 variant polypeptide disclosed herein. Also provided is a method of treating a patient having a disease or a genetic condition that is associated with increased production of free radicals as compared to a normal individual or an increased exposure to free radicals in environmental sources, wherein the method comprises administering the pharmaceutical composition of a SERPIN B1 polypeptide comprising a sequence that is the wild type SERPIN B1 (SEQ ID NO: 1) or a sequence that is at least 90% identical to the wild type SERPIN B1 polypeptide (SEQ ID NO: 1), and a reducing agent disclosed herein.

In some embodiments, the disease or genetic condition is associated with exposure to free radicals present in the environment (e.g. cigarette smoke, vape device emissions) or the increased production of free radicals by enzymes present in innate immune cells, mucosal cells, or glandular cells as compared to a normal individual In some embodiments, the diseases are selected from groups of infectious, autoimmune, respiratory, metabolic, cardiovascular, neurodegenerative or oncology diseases. Infectious diseases include, but are not limited to, pulmonary or systemic diseases such as acute lung injury (ALI), acute respiratory distress (ARDS), pneumonia, bronchiolitis, systemic coagulopathies or hemorrhagic diseases caused by, but not limited to, respiratory syncytial viruses, influenza viruses, coronaviruses, ebola viruses, Pseudomonas aeruginosa and other opportunistic pathogens. Autoimmune diseases include, but are not limited to, type 1 diabetes, rheumatoid arthritis, psoriasis, multiple sclerosis and sterile autoinflammatory diseases (SAID's) that have underlying genetic mutation(s) predisposing patients to recurrent bouts of episodic inflammation. Respiratory diseases include, but are not limited to, allergic asthma, smokers' emphysema, COPD and idiopathic pulmonary fibrosis (IPF). Metabolic diseases include, but are not limited to, type 2 diabetes, insulin resistance, dyslipidemia and cataract formation. Cardiovascular diseases include but are not limited to, atherosclerosis and hypertension. Neurodegenerative diseases include, but are not limited to, Parkinson's and Alzheimer's. Oncology diseases include, but are not limited to, colorectal, pancreatic, prostate, breast, lung and bladder cancers

The oxidation-resistant SERPIN B1 variant polypeptide or composition thereof disclosed herein may be administered by inhalation, intra-tracheally, topically or by injection subcutaneously, intravenously, or intraperitoneally. In some embodiments, the SERPIN B1 variant polypeptide or the wild type SERPIN B1 (SEQ ID NO: 1) is administered at a dose of 0.01 mg to 1000 mg per kg of patient's mass (i.e., 0.01 mg/kg to 1000 mg/kg). In some embodiments, the SERPIN B1 variant polypeptide or the wild type SERPIN B1 is administered in combination with a reducing agent, where the reducing agent is administered in an amount that is sufficient to prevent the oxidation of the C344 of the wild type SERPIN B1 or SERPIN B1 variant polypeptide. In some embodiments, the reducing agent is administered in at a dose of 0.01-100 mg per kilogram of the patient's mass (i.e., 0.01-100 mg/kg).

Also provided herein is a method of producing a wild type SERPIN B1 or a variant polypeptide thereof, the method comprising: expressing the polynucleotide encoding a wild type SERPIN B1 or a variant polypeptide thereof in S. cerevisiae, wherein the SERPIN B1 variant polypeptide comprises an amino acid sequence that is at least 90% identical to SEQ ID NO:1, wherein the SERPIN variant polypeptide comprises an amino acid substitution at residue 344, as compared to the native protein sequence of SEQ ID NO: 1; wherein the SERPIN B1 variant polypeptide is capable of inhibiting the serine protease activity of neutrophil or pancreatic elastase; and wherein the SERPIN B1 variant polypeptide is resistant to oxidation by free radicals. In some embodiments, the S. cerevisiae is protease-deficient.

In some embodiments, the method of expressing the polynucleotide is by introducing a Yeast episomal expression plasmid (Yep) into the S. cerevisiae. In some embodiments, the method of embodiment 19, wherein the polynucleotide is linked to a yeast promoter. In some embodiments, the yeast promoter is an ADH2 promoter. In some embodiments, the polynucleotide is codon optimized for expression in yeast.

In some embodiments, the Serpin B1 variant polypeptide is fused to a Fc portion of an IgG, a single chain variable fragment (scFv) of an antibody, or wherein the Serpin B1 variant polypeptide is pegylated.

### BRIEF DESCRIPTION OF THE DRAWINGS

FIG. 1A-1C. Purification of rhsB1 on hydroxyapatite resin. FIG. 1A and FIG. 1B show the chromatographic results of purification of rhsB1-cys344 (wild-type) (FIG. 1A) and rhsB1-ser344 (variant C344S) (FIG. 1B) on a ceramic hydroxyapatite resin. FIG. 1A shows two main peaks containing rhsB1-cys344 eluting from the resin whereas FIG. 1B shows only one large main peak containing the C344S variant eluting from the resin. These protein peaks were pooled, concentrated and analyzed by 4-20% SDS-PAGE. (C), 4-20% SDS-PAGE on pooled and concentrated rhsB1 peak samples treated without 2-mercaptoethanol (“−2-ME”) (left) or with 2-mercaptoethanol (“+2-ME”) (right). The order of the samples for both the −2-ME group and the +2-ME groups are: 1. Purified rhsB1-C344S; 2. Purified rhsB1-cys344 1^{st} peak (rhsB1M*); 3. Purified rhsB1-cys344 2^{nd} peak (rhsB1D); 4. PAGE-MASTER protein standard plus markers (Genscript; 10, 15, 20, 30, 40, 50, 60, 80 and 120 kDa). The results show that purification of rhsB1-cys344 (wild-type) yields two species of rhsB1. The first major peak eluting in FIG. 1A contains a monomeric form of the protein we have called rhsB1M*. The second major peak eluting in FIG. 1A contains an intermolecular disulfide-bonded dimeric form of the protein that can be reduced to a monomer by the addition of 2-ME. We have called this form of the protein rhsB1D. In contrast, the one large main peak of the C344S variant seen in FIG. 1B contains only monomeric rhsB1 confirming that the intermolecular disulfide bond formation seen with rhsB1 cys-344 (wild-type) is mediated by cys-344.

FIG. 2A-2D. Oxidation of rhsB1 in yeast lysates by N-chlorosuccinimide (NCS). FIG. 2A shows the results of analyzing PPE activity after incubation with 5 uL of yeast lysate containing rhsB1 oxidized with varying concentrations of NCS. Data points are means of triplicate analyses+/−SE. FIG. 2B shows results of 4-20% SDS-PAGE gel and Western blot analysis of rhsB1-containing yeast lysate oxidized with 1 mM NCS; 1. Untreated yeast lysate, 2. Yeast lysate oxidized with 1 mM NCS, 3. Smart dual color pre-stained protein standard (Genscript; 16, 24, 30, 40, 62 and 94 kDa). FIG. 2C shows the results of assaying inhibition of PPE by 1, 3 or 5 uL of oxidized yeast lysate containing rhsB1. Data points are means of triplicate analyses+/−SEM. FIG. 2D shows the results of analyzing the inhibition of bovine α chymotrypsin (BC) by 1, 3 or 5 uL of oxidized yeast lysate containing rhsB1. Data points are means of triplicate analyses+/−SE.

FIG. 3A-3B. Interaction of PPE with rhsB1D and rhsB1M*. FIG. 3A shows the cleavage profile of rhsB1D or rhsB1M* by varying amounts of PPE in the absence of 2-ME as resolved by 4-20% SDS-PAGE. No higher molecular enzyme:inhibitor complexes were observed. Only increasing amounts of lower molecular weight rhsB1 degradation products were observed as the concentration of PPE increased. Lanes 1. PPE only; 2. rhsB1D only; 3-5. PPE:rhsB1D ratios of 1:1,000, 1:100 and 1:10; 6. rhsB1M* only; 7-9. PPE:rhsB1M* ratios of 1:1000, 1:100 and 1:10; M. Markers. In contrast, FIG. 3B shows the results of rhsB1D treated with PPE in the presence of 2-ME. In this example, 2-ME reduced the disulphide bond in rhsB1D to liberate active monomeric rhsB1 (lane 2) which can inhibit and form stable higher molecular complexes with increasing amounts of PPE that are visible on the gel (lanes 3-5): lanes 1. Markers; 2. rhsB1D+2-ME only; 3-5. rhsB1D+2-ME incubated with 0.25, 0.5 or 1.0 molar ratios of PPE; 6. PPE+2-ME only. Samples were incubated for 30 minutes then the reaction stopped and analyzed as described in “experimental procedures”.

FIG. 4A-4B. Impact of 2-ME on inhibition of HNE by rhsB1D and rhsB1M*. FIG. 4A shows the results of analyzing the inhibition of HNE by rhsB1D in the presence of increasing 2-ME concentrations. Two concentrations of rhsB1D (230 or 460 nM) were tested for their ability to inhibit HNE as described in “experimental procedures”; FIG. 4B shows the results of analyzing the inhibition of HNE by rhsB1M* in the presence of increasing 2-ME concentrations. Two concentrations of rhsB1M* (230 or 460 nM) were tested for their ability to inhibit HNE as described in “experimental procedures”. These figures confirm that rhsB1D can be reduced by 2-ME to a monomer which is active as an inhibitor of HNE, confirming and extending the results obtained with PPE. The results show that a 2-ME concentration of at least 40 mM is required to fully reduce the disulfide bond in rhsB1D and restore maximal HNE inhibitory activity. In contrast, the addition of increasing amounts of 2-ME to rhsB1M*did not restore HNE inhibitory activity confirming that this monomeric form of rhsB1 has cys-344 in an irreversibly oxidized state.

FIG. 5A-5D. Interaction of rhsB1D and rhsB1M* with HNE. FIG. 5A shows the inhibition of a fixed concentration of HNE by increasing amounts of rhsB1D in the presence (the sample group “PBS+40 mM+2-ME”) or absence of 2-ME (the sample group “PBS”); FIG. 5B shows the reaction products generated in (A) resolved by gel electrophoresis on a 4-20% SDS-PAG; FIG. 5C shows the inhibition of a fixed concentration of HNE by increasing amounts of rhsB1M* in the presence or absence of 2-ME; FIG. 5D shows the reaction products generated in FIG. 5C resolved by gel electrophoresis on a 4-20% SDS-PAG. The numeral values, 0.7, 1.4, 2.1 in FIG. 5B represent the molar ratios of rhB1D to HNE. The numeral values, 0.7, 1.4, 2.1 in FIG. 5D represent the molar ratios of rhB1M* to HNE. The results confirm that 2-ME is required to reduce rhsB1D into an active monomer that can inhibit HNE and form SDS-stable complexes. In contrast, rhsB1M* was unable to efficiently complex with and inhibit HNE in the presence of 2-ME.

FIG. 6A-6D. Interaction of chymotrypsin with rhsB1D and rhsB1M*. FIG. 6A shows the inhibition of a fixed concentration of chymotrypsin by increasing amounts of rhsB1D in the presence (the sample group “PBS+40 mM+2-ME”) or absence of 2-ME (the sample group “PBS”); FIG. 6B shows the reaction products generated in FIG. 6A resolved by 4-20% SDS-PAGE; FIG. 6C shows the inhibition of a fixed concentration of chymotrypsin by increasing amounts of rhsB1M* in the presence or absence of 2-ME; FIG. 6D shows the reaction products generated in (C) in the absence of 2-ME as resolved by 4-20% SDS-PAGE. The “Molar Ratio I:E” represents the molar ratio of rhB1D to chymotrypsin (FIG. 6B) or the molar ratio of rhB1M* to chymotrypsin (FIG. 6C). The results show that in the presence or absence of 2-ME rhsB1D can effectively inhibit chymotrypsin at near or below equimolar ratios. However, dissociation of rhsB1D into its active monomer form with 2-ME makes it 2-fold more effective at inhibiting chymotrypsin. In contrast, rhsB1M* can also inhibit chymotrypsin in the presence or absence of 2-ME but it is 3-5 times less effective compared to rhsB1D and its activity is unaffected by 2-ME.

FIG. 7A-7C. Interaction of cathepsin G with rhsB1D and rhsB1M*. FIG. 7A shows the inhibition of a fixed concentration of human cathepsin G by increasing amounts of rhsB1D and rhsB1M* in the presence or absence of 2-ME. FIG. 7B shows the SDS-PAGE analysis of the reaction products generated by the interaction of rhsB1D and cathepsin Gin the presence or absence of 2-ME. FIG. 7C shows the SDS-PAGE analysis of the reaction products generated by the interaction of rhsB1M* and cathepsin G in the absence of 2-ME. The “Molar Ratio I:E” represents the molar ratio of rhB1D to cathepsin G (FIG. 7B) or the molar ratio of rhB1M* to cathepsin G (FIG. 7C). The results show that in the presence or absence of 2-ME rhsB1D can effectively inhibit cathepsin G at or slightly above near equimolar ratios. However, dissociation of rhsB1D into its active monomer form with 2-ME makes it 2-fold more effective at inhibiting cathepsin G. In contrast, rhsB1M* can also inhibit cathepsin G in the presence or absence of 2-ME but it is 3-5 times less effective compared to rhsB1D and its activity is unaffected by 2-ME.

FIG. 8. Potential Programmed Cell Death Pathways activated by oxidation of Cys-344 in human Serpin B1. This figure shows the potential relationship between oxidation of C344 in Serpin B1 and the activation of various programmed cell death pathways. The initial step involves the oxidative inactivation of C344 on the exposed reactive site loop (RSL—depicted as an extended blue rectangle sticking out of sB1) of serpin B1 (sB1). Oxidized sB1 is devoid of elastase inhibitory activity and has reduced inhibitory activity against cathepsin G and proteinase 3 (CatG, PR3). Oxidized sB1 no longer can inhibit elastase and instead, gets cleaved in the RSL inducing a structural change in the protein (depicted as a square blue box from which the “red” CARD domain of procaspases 1,4, and 5 has now been expelled) which i) destroys all residual protease inhibitory activity that sB1 has and ii) allows the self-association and activation of caspases 1, 4, and 5). Increased enzyme activity (elastase, CatG, PR3, caspases) then acts on downstream effector proteins (e.g. gasdermin D (GSDMD), procaspase 3 and pro-interleukins (e.g. proIL1-beta)) to allow a number of different types of programmed cell death pathways to proceed such as (Pyroptosis, NETosis and Necrosis; Choi et al., 2019; Pappayannopoulos et al., 2010; Burgener at al., 2019)

FIG. 9. Protein sequencing data for PPE cleaved rhsB1M* and rhsB1D. (described in Examples 1 and 5). This data shows which peptide bonds are cleaved in oxidized sB1.

FIG. 10. Cysteine residues in Clade B serpin reactive site loops. This figure shows the placement of C344 within the reactive site loop in human serpin B1 (MNEI) compared to the 4 other highly homologous human clade B (intracellular) serpins that also have cysteine residues in their reactive site loops.

FIG. 11. sB1 RSL protein sequences in different species. This figure shows the alignment of serpin B1 reactive site protein sequences for different species. Note that C344 is highly conserved in humans, rodents and monkeys.

FIG. 12. shows that the wild-type human serpin B1 (C344) was rapidly inactivated as an elastase inhibitor by reactive oxygen and nitrogen species (ROS/RNS) but not H2O2.

FIG. 13 shows that the oxidation of wild-type human serpin B1 (C344) reduced its ability to inhibit other proteases. rhsB1M*P represents purified peroxynitrite inactivated wild-type rhsB1; rhsB1-CL represents rhsB1 WT that has been cleaved and inactivated by PPE.

FIG. 14 shows the effect of amino acid substitutions at C344 in human Serpin B1 on elastase inhibition.

FIG. 15 shows that the C344S human serpin B1 variant was cleaved and inactivated by PPE at high enzyme:inhibitor ratios rather than forming stable complexes.

FIG. 16 shows that the human serpin B1 C344A variant retained elastase and chymotrypsin inhibitory activity in the presence of peroxynitrite.

FIG. 17 shows that the wild-type human serpin B1 but not the C344A variant was rapidly inactivated by myeloperoxidase (MPO) generated free radicals.

### DETAILED DESCRIPTION OF THE INVENTION

### Terminology

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

All numerical designations, e.g., pH, temperature, time, concentration, amounts, a molecular weight, including ranges, are approximations which are varied (+) or (−) by increments of 0.1 or 1.0, where appropriate. It is to be understood, although not always explicitly stated, that all numerical designations may be preceded by the term “about.” It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” is intended to mean that the compositions and methods include the recited elements, but do not exclude others. “Consisting essentially of” when used to define compositions and methods, refers to the specified materials or steps and those that do not materially affect the basic and novel characteristic(s) of the claimed invention. “Consisting of” shall mean excluding more than trace amounts of other ingredients and substantial method steps recited. Embodiments defined by each of these transition terms are within the scope of this invention.

The term “rhsB1,” disclosed herein refers to the native human SERPIN B1 polypeptide (SEQ ID NO: 1) that is produced in non-human host cells.

The term “SERPIN B1 polypeptide,” or “SERPIN B1,” refers to a native (also referred to as “wild type”) SERPIN B1 polypeptide having the sequence of SEQ ID NO: 1, or a variant thereof (i.e., a SERPIN B1 variant polypeptide).

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three dimensional structure and may perform any function, known or unknown. The following are non limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non nucleotide components. A polynucleotide can be further modified after polymerization, such as by conjugation with a labeling component. The term also refers to both double and single stranded molecules. Unless otherwise specified or required, any embodiment of this invention that is a polynucleotide encompasses both the double stranded form and each of two complementary single stranded forms known or predicted to make up the double stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule.

The term “percent identity” refers to sequence identity between two peptides or between two nucleic acid molecules. Percent identity can be determined by comparing a position in each sequence which may be aligned for purposes of comparison. When a position in the compared sequence is occupied by the same base or amino acid, then the molecules are identical at that position. As used herein, the phrase “homologous” or “variant” nucleotide sequence,” or “homologous” or “variant” amino acid sequence refers to sequences characterized by identity, at the nucleotide level or amino acid level, of at least a specified percentage. Homologous nucleotide sequences include those sequences coding for naturally occurring allelic variants and mutations of the nucleotide sequences set forth herein. Homologous nucleotide sequences include nucleotide sequences encoding for a protein of a mammalian species other than humans. Homologous amino acid sequences include those amino acid sequences which contain conservative amino acid substitutions and which polypeptides have the same binding and/or activity. In some embodiments, a homologous nucleotide or amino acid sequence has at least 60% or greater, for example at least 70%, or at least 80%, at least 85% or greater, with a comparator sequence. In some embodiments, a homologous nucleotide or amino acid sequence has at least 90%, 91%, 92%, 93%, 94%, 95%, 96%, 97%, 98% or 99% identity with a comparator sequence. In some embodiments, a homologous amino acid sequence has no more than 15, nor more than 10, nor more than 5 or no more than 3 conservative amino acid substitutions. Percent identity can be determined by, for example, the Gap program (Wisconsin Sequence Analysis Package, Version 8 for UNIX, Genetics Computer Group, University Research Park, Madison Wis.), using default settings, which uses the algorithm of Smith and Waterman (Adv. Appl. Math., 1981, 2, 482-489).

The term “express” refers to the production of a gene product. The term “transient” when referred to expression means a polynucleotide is not incorporated into the genome of the cell.

The term “vector” refers to a non-chromosomal nucleic acid comprising an intact replicon such that the vector may be replicated when placed within a permissive cell, for example by a process of transformation. A vector may replicate in one cell type, such as bacteria, but have limited ability to replicate in another cell, such as mammalian cells. Vectors may be viral or non-viral. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “normal individual,” as used herein, refers to a healthy, non-smoking individual.

The term “associated with,” with regard to the relationship between a disease or genetic condition and free radicals, refers to that the disease or genetic condition is at least in part resulted from exposure to a high level of free radicals in the environment or inside the body, or that the disease or genetic condition causes the increased production of free radicals in the body as compared to a normal individual.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. The term “administering” or “administration” of a monoclonal antibody or a natural killer cell to a subject includes any route of introducing or delivering the antibody or cells to perform the intended function. Administration can be carried out by any route suitable for the delivery of the cells or monoclonal antibody. Thus, delivery routes can include intravenous, intramuscular, intraperitoneal, or subcutaneous deliver.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering a therapeutically effective amount of a fusion protein described herein.

The term “therapeutically effective amount” or “effective mount” includes an amount or quantity effective, at dosages and for periods of time necessary, to achieve the desired therapeutic or prophylactic result.

As used herein, the term “substantially the same”, when referring to the enzymatic inhibition activity, for example, neutrophil elastase inhibition activity, refers to that the two measurements of inhibition activity are no more than 25%, no more than 20%, no more than 15% different, no more than 10%, no more than 8%, or no more than 5% different from each other.

### INTRODUCTION

Human serpin B1 (hsB1) was first identified in 1985 as a fast acting elastase inhibitor present in high concentrations in human monocytes cultured in vitro and subsequently in macrophages and neutrophils (Remold-O'Donnell et al., J. Exp. Med. 162, 2142-2155 (1985); Remold-O'Donnell et al., J. Exp. Med. 169, 1071-1086 (1989), 2). It has a molecular weight of approximately 42 kDa and is a member of the clade B branch of the serpin superfamily of proteins that do not possess classical secretion signals (Remold-O'Donnell et al., Proc. Natl. Acad. Sci., USA 89, 5635-5639 (1992)). Sequence alignments with other serpins identified Cys-344 as the putative P1 residue on the reactive site loop (RSL) responsible for the observed EIA. N-terminal sequence data derived from elastase-hsB1 complexes and the sensitivity of the proteins EIA to the alkylating agent iodoacetamide subsequently confirmed this assignment (Remold-O'Donnell et al., J. Exp. Med. 169, 1071-1086 (1989); Cooley et al. Biochemistry 40, 15762-15770 (2013)). A number of serpins have been demonstrated to utilize overlapping reactive sites to efficiently inhibit different classes of proteinases. This is also true for hsB1 which utilizes Phe-343 to inhibit chymotrypsin-like proteases including bovine chymotrypsin, cathepsin G (catG), mast cell chymase, granzyme H (GmzH) and prostate specific antigen (PSA)—in addition to Cys-344 which it also utilizes to inhibit neutrophil proteinase 3 (Cooley et al.; Wang et al., J. Immunol. 190, 1319-1330 (2013)).

Serpin B1 exhibits a number of protective anti-inflammatory roles in vivo. Recombinant hsB1 has been used prophylactically to protect rat lungs against injury mediated by pro-inflammatory cystic fibrosis airway secretions, suppress bacterial proliferation in a mouse model of P. aeruginosa lung infection and ameliorate post-operative acute lung injury in a rat model of liver transplantation (Cooley et al. (1998)). In 2007, Benerafa et al. knocked out the gene for the murine homolog (sb1a) of human sB1 and demonstrated the critical role it plays in regulating excessive inflammatory responses during bacterial infection (Benerafa et al. (2007)). In 2011 Gong et al, used the same model to demonstrate that sb1a also protected against the excessive inflammation induced by pulmonary influenza without affecting viral clearance (Gong et al., (2011)). Further studies by the groups of Benarafa and O'Donnell have revealed that sb1a protects neutrophils in the bone marrow and prevents programmed necrosis in neutrophils and monocytes via the specific inhibition of cathepsin G (Benerafa et al., (2011)). In 2012, Farley et al, discovered that sB1 can regulate the formation of neutrophil extracellular traps (NETs) induced by multiple different stimuli—a process in part dependent on the production of ROS by myeloperoxidase (MPO) (Choi et al. (2019)). In this role sb1a was observed to migrate from the cytoplasm of neutrophils into the nucleus but the mechanism driving this translocation and its nuclear target(s) are currently unknown (Farley et al. (2012)). In addition, sb1a reportedly regulates the expansion of Th17 phenotype T-cells via inhibition of cysteine cathepsins, most prominently cathepsin L (Zhao et al. (2014)). More recently, El Ouaamari et al., identified sB1 as a factor that promotes compensatory (3-cell responses to insulin resistance by inducing pancreatic β-islet cell proliferation via modulation of proteins in growth and survival pathways (El Ouaamari et al. (2016)) while Choi et al identified a CARD (caspase recruitment domain) binding motif (CBM) in Serpin B1 that is responsible for limiting the activation of inflammatory caspases 1, 4, 5 and 11 and preventing pyroptosis (Choi et al. (2019)).

Others have suggested sB1 to play a direct role in apoptosis. In 1998, Torriglia et al. partially sequenced a ubiquitous intracellular cation-independent acidic endonuclease called DNase II and found it to be homologous with the protein sequence of porcine sB1 (Torriglia et al. (1998)). Since then, this group has published a number of papers suggesting that, upon cleavage by elastase or some other protease involved in apoptosis, sB1 undergoes a structural rearrangement to unmask a latent endonuclease activity in a molecule which they call L-DNase II—a truly unique and surprising activity for a serpin if confirmed (Padron-Barthe et al. (2007)).

In this application, we disclose that the exposed physical location of Cys-344 in sB1 would render it sensitive to reactive free radical species (e.g., ROS or RNS) produced during inflammatory or redox mediated signaling events. The present application discloses that maintenance of Cys-344 in a reduced state is required for sB1 to efficiently inhibit elastase and demonstrate that oxidation of Cys-344 can produce post-translationally modified (PTM) forms of sB1 with altered protease inhibitory activities. During the early stages of inflammation and perhaps other redox mediated cell-signaling events, the control functions of SERPIN B1 may be down-regulated by certain types of free radicals allowing inflammation to proceed. The present application thus provides methods and compositions comprising a SERPIN B1 variant polypeptide, in which The SERPIN B1 variant polypeptide possesses neutrophil elastase inhibitory activity and the neutrophil elastase inhibition activity is resistant to oxidation by free radicals. In some cases, the SERPIN B1 variant polypeptide has an amino acid sequence that is at least 90% identical to SEQ ID NO: 1. These SERPIN B1 variant polypeptides can be used to treat patients having a disease or genetic condition that is associated with the increased production of free radicals in neutrophils, monocytes as compared to a normal individual.

A. The Elastase Inhibition Activity of SERPIN B1 Requries Maintaining C344-SH in a Reduced State

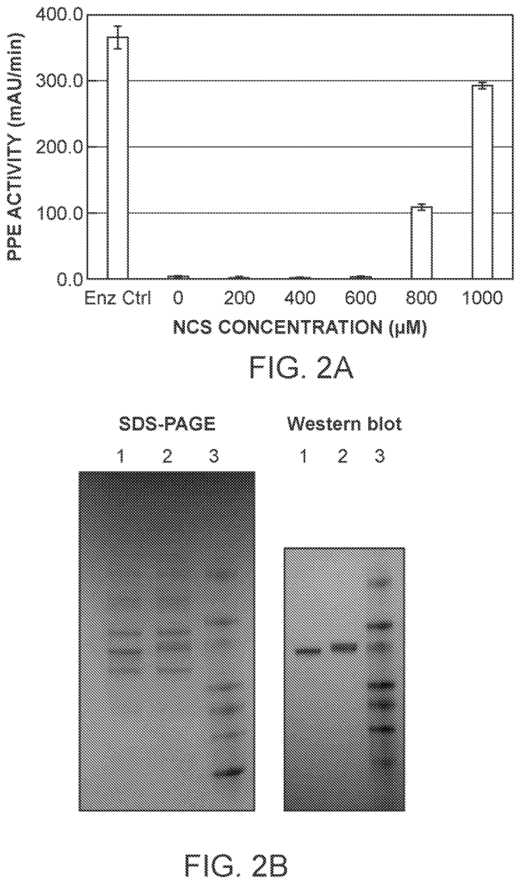

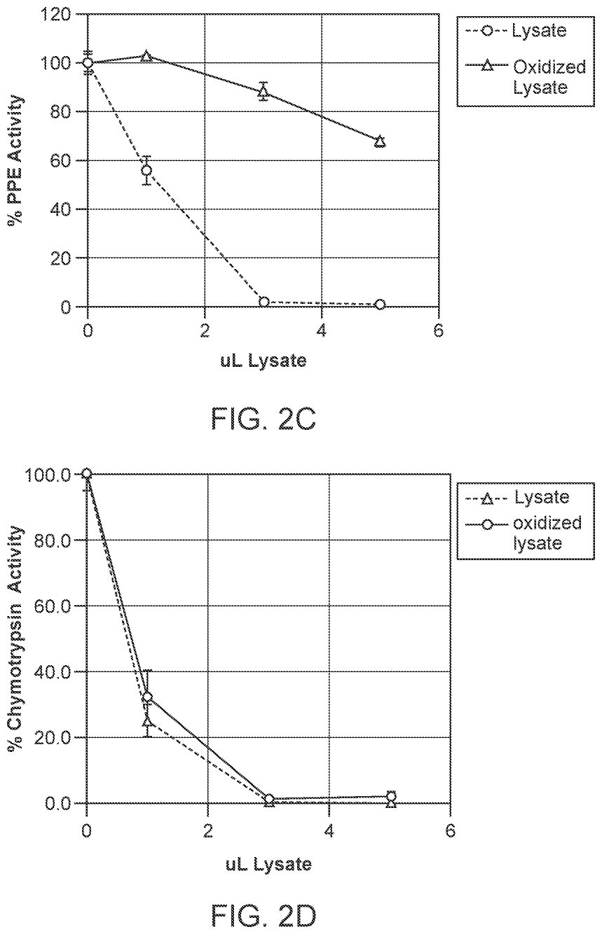

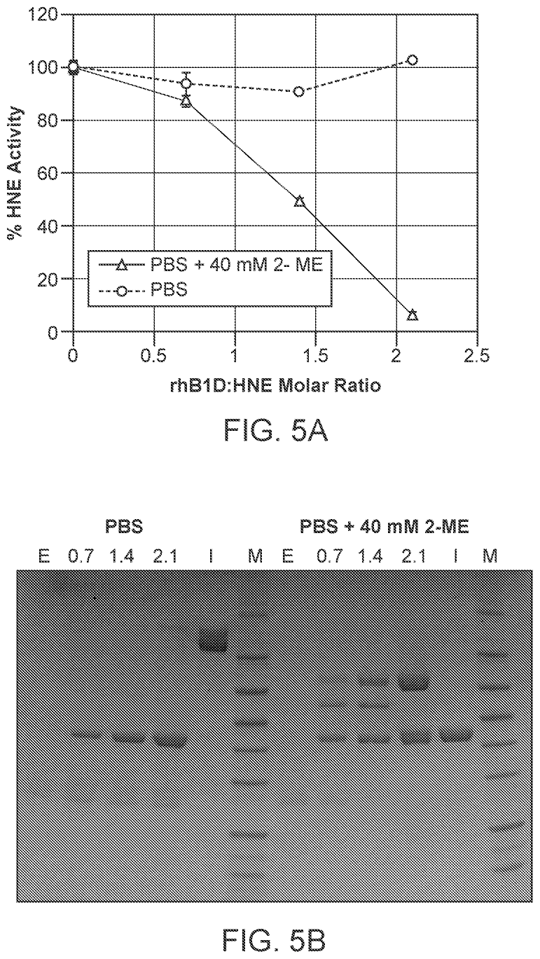

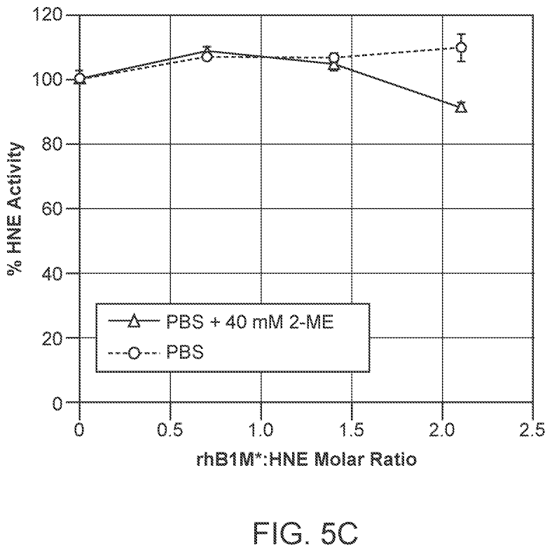

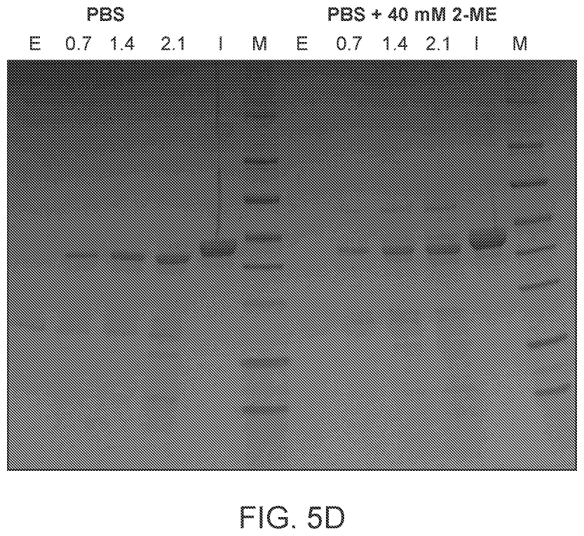

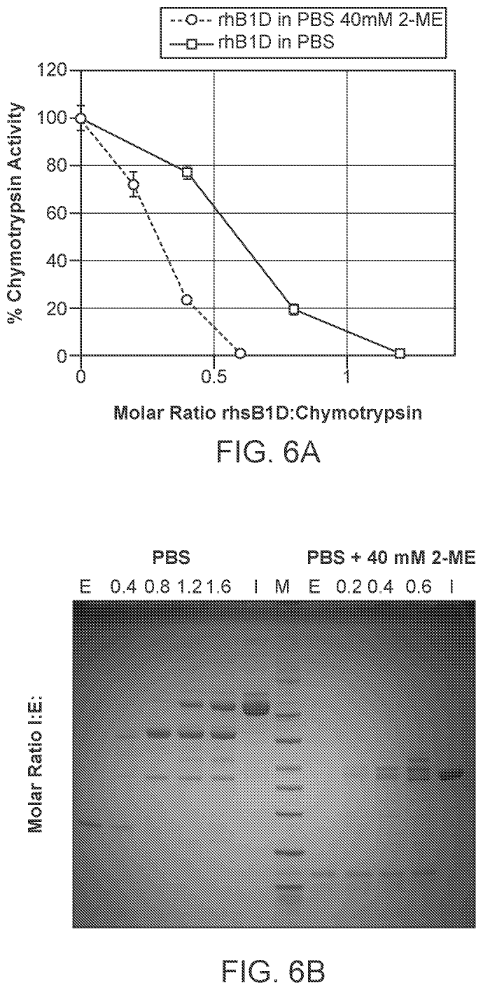

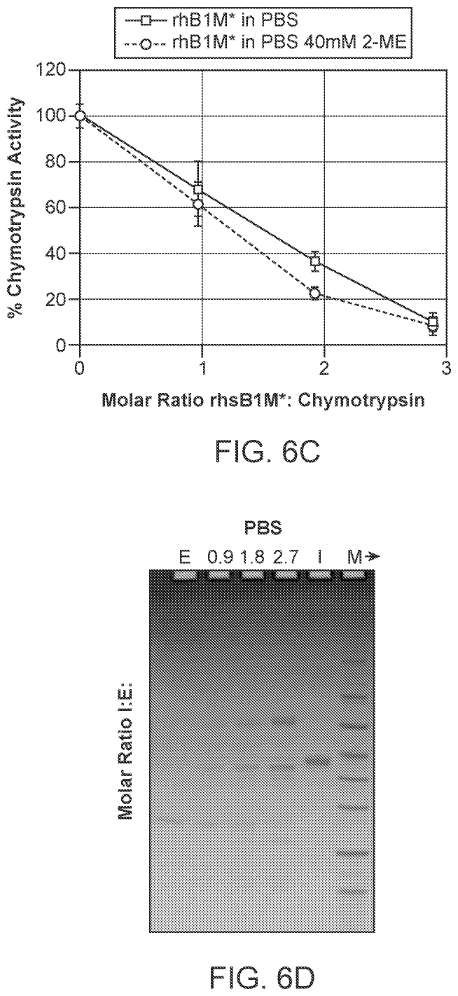

Here we provide evidence that the structurally exposed Cys-344 (Wang et al., J. Immunol. 190, 1319-1330 (2013), is subject to post-translational modifications (PTM's) that convert sB1 from an inhibitor to substrate of elastase which results in in the loss of elastase inhibition activity and can lead to the complete loss of all protease inhibitory activity. See Examples 3, 10 and 11. The recombinant human serpin B1 (rhsB1) produced intracellularly in yeast inhibits both elastase and chymotrypsin (EIA and CIA) but EIA is sensitive to rapid inactivation by the oxidizing agents N-chlorosuccinimide, peroxynitrite and sodium hypochlorite and free radicals generated by myeloperoxidase (FIGS. 2, 12 and 17). We further show that purification of rhsB1 in the absence of reducing agents results in the progressive and specific loss of EIA (FIG. 5A), but not CIA (FIG. 6A), concomitant with the formation of two unique higher molecular weight forms of rhsB1; a modified monomer (rhsB1M*) and a dimer (rhsB1D) created via an intermolecular disulfide bond formed between Cys-344 in respective rhsB1 monomers. Unlike fully reduced rhsB1, rhsB1M* and rhsB1D are good elastase substrates that are rapidly and catalytically cleaved at multiple adjacent sites within the reactive site loop (RSL; Gly339-Ile340, Ala341-Thr342 and Thr342-Phe343). Furthermore, even though rhsB1M* and rhsB1D retain CIA their respective efficiencies of inhibition are reduced significantly.

These findings confirm that hsB1 is capable of inhibiting both elastase and chymotrypsin-like proteases under conditions where maintenance of a reduced Cys-344 prevails. However, we propose that during inflammation or redox-mediated cell signaling events involving certain types of reactive oxygen species (ROS) or reactive nitrogen species (RNS), PTM's of Cys-344 may be a key event allowing inflammatory pathways to proceed via increased elastase, cathepsin G and proteinase 3 activity. The conversion of hsB1 to a cleaved inactive (R) form by elastase will completely inactivate all direct protease inhibitory activity mediated by the reactive site loop and may also disrupt other regulatory domain(s) within the proteins tertiary structure (e.g. CBM) allowing the activation/amplification of other pro-inflammatory pathways.

B. Oxidation-Resistant SERPIN B1 Variants

The disclosure provides a SERPIN B1 variant polypeptide possessing neutrophil elastase inhibition activity and the neutrophil elastase inhibitory activity is resistant to oxidation by free radicals. Neutrophil (and pancreatic) elastase inhibitory activity of the native SERPIN B1 is dependent on the reduced form of C344 (“C344 dependent elastase inhibition activity”). The C344 dependent elastase inhibitory activity of SERPIN B1 is susceptible to oxidation by free radicals because the oxidation of the C344 of the SERPIN B1 results in a significant reduction or a complete loss of elastase inhibitory activity. The term “free radical” or “radical,” or “reactive free radical species” typically refers to a molecule with an unpaired electron and capable of high reactivity. Radicals have extremely high chemical reactivity and when generated in excess or not appropriately controlled, may inflict damage upon cells. Free radicals disclosed in this disclosure refers to any cysteine reactive free radical species, e.g., a reactive free radical species that can oxidize C344 of the native SERPIN B1 (SEQ ID NO: 1) and decrease its elastase inhibitory activity. The decrease in the elastase inhibitory activity may be at least 20%, at least 30%, at least 40%, at least 50%, or at least 60% as compared to the native SERPIN B1, the C344 of which is unoxidized. These free radicals may include, but are not limited to, those derived from oxygen (“reactive oxygen species”) or those derived from nitrogen (“reactive nitrogen species”). Oxidation of cysteine by the free radicals may produce a variety of oxidation products, including, e.g., S-nitrosocysteine, cysteine sulfenic, sulfinic and sulfonic acids, disulfides and persulfides. As a result, oxidation of cysteine by free radicals may have toxicological implications in a number of diseases, such as emphysema and cancer, and impair the body's anti-bacterial and anti-viral defenses.

Free radicals are present in, or may be induced by exogenous sources such as smoke and pollution particles and can be produced by endogenous sources in response to pathogens such as viruses or bacteria, or genetic conditions predisposing an individual to “sterile” autoinflammatory diseases (SAID's). Endogenous ROS and RNS are produced by enzymes (e.g. peroxidases and nitric oxide synthases) that are resident in, or secreted from innate immune cells (e.g. neutrophils, eosinophils, macrophages, monocytes), mucosal cells (e.g. lung airway mucosa, intestinal mucosa), and glandular cells (e.g. thyroid, mammary and salivary).

Non-limiting examples of ROS include hypochlorous acid, hypochlorite, N-chlorosuccinimide, hydrogen peroxide, and sodium hypochlorite. Non-limiting examples of RNS include nitric oxide (NO). Some agents can be both ROS and RNS, for example, peroxynitrite. Other examples of reactive free radical species include, but not limited to, those as described in Griendling et al. (2016), Measurement of Reactive Oxygen Species, Reactive Nitrogen Species, and Redox Dependent Signaling in the Cardiovascular System Circulation Research, Vol. 119, No. 5. As non-limiting examples, neutrophils produce myeloperoxidase which converts H2O2 and NaCl into the ROS hypochlorous acid and hypochlorite (HOCl, ⁻OCl)—much more potent free radicals which are antibacterial and an important part of the host defense mechanism but can also damage cell membranes, DNA and proteins (Klebanoff, S. J. (2005). Myeloperoxidase: Friend and Foe. J. Leucocyte Biology. 77: 598-625). Macrophages produce inducible nitric oxide synthase 2 (iNOS) which produces large amounts of nitric oxide (NO). H2O2 and NO combine to form the very powerful RNS peroxynitrite (ONOO⁻) which is also an important part of the host defense mechanism but also damages membranes, DNA and proteins (Pacher P, Beckmann J S and Liaudet L. (2007). Nitric Oxide and Peroxynitrite in Health and Disease. Phsiol. Rev., 87(1): 315-424).

Mucosal and glandular cells produce lactoperoxidase which catalyzes the conversion of thiocyanate (SCN) into hypothiocyanite (OSCN) in the presence of H2O2. Hypothiocyanite has potent antibacterial activities and appears non-toxic to human cells (Day B J. (2019). The science of licking your wounds: Function of oxidants in the innate immune system. Biochem Pharmacol., 163: 451-457).

The SERPIN B1 variant polypeptides disclosed herein can retain their neutrophil or pancreatic elastase inhibition activity even in the presence of free radicals. Elastase inhibition activity of the variant polypeptides can be tested using methods well known in the art. For example, elastases can be incubated with the SERPIN B1 variant polypeptide, followed by adding an elastase substrate. The elastase cleaves the substrate to produce a colorimetric or fluorescent signal, which can be detected using a suitable device. One exemplary substrate is Succ-AAPV-pNA. Similar assays can be performed with a SERPIN B1 variant polypeptide that has been treated with a free radical (ROS or RNS) or an enzyme or other agent that produces free radicals, e.g., peroxynitrite, hypochlorous acid, or myeloperoxidase, to assess its elastase inhibition activity. The elastase inhibition activity of SERPIN B1 variant polypeptides after being exposed to free radicals is substantially the same as that of the SERPIN B1 variant polypeptides before the exposure. One illustrative example of the elastase inhibition assay is described in Example 1.

The variant polypeptide(s) has an amino acid sequence that is at least 90%, at least 91%, at least 92%, at least 93%, at least 95%, at least 96%, at least 97%, %, at least 98%, at least 99% identical to SEQ ID NO: 1 over the full length sequence of SEQ ID NO: 1, and the variant polypeptide is also able to inhibit the protease activity of an elastase (e.g., human neutrophil elastase or a pancreatic elastase). In some embodiments, the variant polypeptide comprises a single amino acid substitution that is selected from the group consisting of C344A, C344V, and C344G and as relative to the native human SERPIN B1 (SEQ ID NO: 1). In some embodiments, the variant polypeptide comprises the sequence of SEQ ID NO: 2, SEQ ID NO:3, or SEQ ID NO: 4.

Sequence identity or similarity disclosed herein may be determined using standard techniques known in the art, including, but not limited to, the local sequence identity algorithm of Smith & Waterman J. of Mol. Biol. Vol. 147, Issue 1:195-197 (1981); the sequence identity alignment algorithm of Needieman & Wunsch; the search for similarity method of Pearson & Lipman; the computerized implementations of these algorithms (GAP, BESTFIT, FASTA, BLAST, Clustal Omega, and TFASTA in the Wisconsin Genetics Software Package, Genetics computer Group, 575 Science Drive, Madison, Wis.); or the Best Fit sequence program described by Devereux et al. Nucleic Acids Res. 12:387-95 (1984), preferably using the default settings. In one embodiment, computerized implementations of the BLAST 2.0 algorithm described in Altschul et al., 1990, J. Mol. Biol. 215:403-410 (using default parameters) may be used to determine the sequence identity.

Sequence identity can also be determined by inspection of the sequences. For example, the sequence identity between sequence A and sequence B, aligned using the software above or manually, can be determined by dividing the sum of the residue matches between sequence A and sequence B by the result of the length of sequence A, minus the number of gap residues in sequence A, minus the number of gap residues in sequence B, times one hundred.

SERPIN B1 variant polypeptides can be generated by modifying the native polypeptide (SEQ ID NO: 1) according to methods well-known to the skilled in the art. Such methods include, but are not limited to, mutagenesis by PCR, which uses primers designed to contain desired changes; nested primers to mutate a target region; and inverse PCR, which amplifies a region of unknown sequence using primers orientated in the reverse direction. Many other mutation and evolution methods are also available and expected to be within the skill of a person of ordinary skill in the relevant art.

The polynucleotides encoding the SERPIN B1 variant polypeptides described herein may also be chemically synthesized in accordance with the desired sequence by a known synthesis process. These sequences can be cloned into an expression vector using well-established cloning procedures, as further described below.

Chemical or enzymatic alterations of expressed polynucleotides and polypeptides can be performed. For example, sequences can be modified by the addition of lipids, sugars, peptides, organic or inorganic compounds, by the inclusion of modified nucleotides or amino acids, or the like using standard methods. Accordingly, the present invention provides for modification of any of the SERPIN B1 variant polynucleotides or polypeptides by mutation, chemical or enzymatic modification, or other available methods, as well as for the products produced by practicing such methods, e.g., using the sequences herein as a starting substrate for the various modification approaches.

C. Pharmaceutical Compositions Comprising SERPIN B1 or SERPIN B1 Variants

This disclosure also provides pharmaceutical compositions comprising a native SERPIN B1 or a SERPIN B1 variant polypeptide disclosed herein, and one or more pharmaceutically acceptable carriers. The SERPIN variant polypeptide possesses neutrophil elastase inhibition activity. In some embodiments, the neutrophil elastase inhibition activity is resistant to oxidation by free radicals. In some embodiments, the pharmaceutical composition comprises a native SERPIN B1, or a SERPIN B1 variant polypeptide, which has an amino acid sequence that is at least 90%, at least 90%, at least 91%, at least 92%, at least 93%, at least 95%, at least 96%, at least 97%, %, at least 98%, at least 99% identical to SEQ ID NO: 1. In some embodiments, the variant polypeptide comprises a single amino acid substitution that is selected from the group consisting of C344G, C344A, and C344V as compared to the native human SERPIN B1 (SEQ ID NO: 1). In some embodiments, the variant polypeptide comprises the sequence of SEQ ID NO: 2, SEQ ID NO:3, or SEQ ID NO: 4.

In some embodiments, the pharmaceutically acceptable carrier is a reducing agent (e.g., N-acetylcysteine (NAC)), which is capable of preventing C344 of SERPIN B1 polypeptide from being oxidized by the free radicals. In some embodiments, the pharmaceutical composition comprises the native SERPIN B1, which has the sequence of SEQ ID NO: 1 and the reducing agent. In some embodiments, the pharmaceutical composition comprises a variant SERPIN B1, which has an amino acid sequence that is at least 90%, at least 90%, at least 91%, at least 92%, at least 93%, at least 95%, at least 96%, at least 97%, %, at least 98%, at least 99% identical to SEQ ID NO: 1 over the full length sequence of SEQ ID NO: 1, and the variant polypeptide is also able to inhibit the protease activity of a neutrophil elastase (e.g., a human neutrophil elastase) or a pancreatic elastase (e.g. a human pancreatic elastase).

Other pharmaceutically acceptable carriers, excipients, or stabilizers may also be used at suitable dosages and concentrations. These pharmaceutically acceptable carriers, excipients, or stabilizers include, but are not limited to, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the SERPIN B1 polypeptide or a variant thereof to a patient in need thereof.

The amount of the reducing agent used in the pharmaceutical composition may vary, and the amount should be sufficient to prevent oxidation of C344 of the wild type SERPIN B1 polypeptide or SERPIN B1 variant polypeptides.

D. Treating Diseases with Oxidation-Resistant SERPINS

The SERPIN B1 variant polyptides or pharmaceutical composition comprising thereof can be used to treat a patient having a disease or genetic condition associated with exposure to high levels of free radicals or increased exposure to the free radicals present in environmental sources (e.g. air pollutants including tobacco smoke, vape device emissions). The SERPIN B1 variant polyptides or pharmaceutical composition comprising thereof can also be used to treat a disease or genetic condition that is associated with increased production of free radicals, by e.g., activated endogenous enzymes (e.g. peroxidases or nitric oxide synthases) resident in innate immune cells (e.g. neutrophils, monocytes, macrophages, eosinophils) or tissues and organs as compared to a normal individual.

Non-limiting examples of these diseases are selected from groups of infectious, autoimmune, respiratory, metabolic, cardiovascular, neurodegenerative or oncology diseases. Infectious diseases include, but are not limited to, pulmonary or systemic diseases such as acute lung injury (ALI), acute respiratory distress (ARDS), pneumonia, bronchiolitis, systemic coagulopathies or hemorrhagic diseases caused by, but not limited to, respiratory syncytial viruses, influenza viruses, coronaviruses, ebola viruses, Pseudomonas aeruginosa and other opportunistic pathogens. Autoimmune diseases include, but are not limited to, type 1 diabetes, rheumatoid arthritis, psoriasis, multiple sclerosis and sterile autoinflammatory diseases (SAID's) that have underlying genetic mutation(s) predisposing patients to recurrent bouts of episodic inflammation. Respiratory diseases include, but are not limited to, allergic asthma, smokers' emphysema, COPD and idiopathic pulmonary fibrosis (IPF). Metabolic diseases include, but are not limited to, type 2 diabetes, insulin resistance, dyslipidemia and cataract formation. Cardiovascular diseases include but are not limited to, atherosclerosis and hypertension. Neurodegenerative diseases include, but are not limited to, Parkinson's and Alzheimer's. Oncology diseases include, but are not limited to, colorectal, pancreatic, prostate, breast, lung and bladder cancers. Examples of diseases that are associated with free radicals are also described in Maddu, Disease Related To Types Of Free Radicals, (2019), DOI: 10.5772/intechopen.82879, the entire content of which is herein incorporated by reference.

The pharmaceutical compositions of the wild type SERPIN B1 or SERPIN B1 variant polypeptides may be administered to a subject at a therapeutically effective dose to treat a disease or a genetic condition as described above. The pharmaceutical compositions can be administered by, e.g., inhalation, intra-tracheally, topically or by injection subcutaneously, intravenously, or intraperitoneally.

Generally administered dosage will be one that is effective to achieve the desired therapeutic effect. One of ordinary skill in the art understands that the dose administered will vary depending on a number of factors, including, but not limited to, the subject's body weight, age, individual condition, surface area or volume of the area to be treated, and/or on the form of administration. The size of the dose also will be determined by the existence, nature, and extent of any adverse effects that accompany the administration of a particular compound in a particular subject. Preferably, the smallest dose and concentration required to produce the desired result should be used. Dosage should be appropriately adjusted for children, the elderly, debilitated patients, and patients with cardiac and/or liver disease. Further guidance can be obtained from studies known in the art using experimental animal models for evaluating dosage. In some embodiments, the administered dosage is one that delivers an amount of the native SERPIN B1 (SEQ ID NO: 1) or SERPIN B1 variant polypeptide that range from 0.01-1000 mg/kg, e.g., 0.1-500 mg/kg, 0.5-100 mg/kg, 1.0-50 mg/kg, or from 1.0-25 mg/kg. In some embodiments, the native SERPIN B1 or the SERPIN B1 variant polypeptide as disclosed herein is administered in combination with (e.g., simultaneously or sequentially) a reducting agent disclosed herein. In some embodiments, the reducing agent (e.g., NAC) may be delivered in an amount of 0.01-100 mg per kilogram of the patient's mass.

Optimal dosing schedules can be calculated from measurements of agent accumulation in the body of a subject. In general, dosage may be given once or more daily, weekly, or monthly. Persons of ordinary skill in the art can easily determine optimum dosages, dosing methodologies, and repetition rates. In some embodiments, the compositions of the invention are administered one or more times a day, e.g., 1, 2, 3, 4, 5, 6, 7, 8, 9, 10, or more times a day. In some embodiments, the compositions of the invention are administered for about 1 to about 31 days, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the compositions of the invention are administered for at least 1 day. In other embodiments, the compositions of the invention are administered for one or more weeks, e.g., 1, 2, 3, 4, 5, 6, 7, 8, 9, 10, 11, 12, 13, 14, or more weeks. In yet other embodiments, the compositions are administered for one or more months, e.g., 1, 2, 3, 4, 5, 6, 7, 8, 9, 10, 11, 12, or more months.

To achieve the desired therapeutic effect, the compositions of the invention may be administered for multiple days at the therapeutically effective daily dose. Thus, therapeutically effective administration of the compositions of the invention to treat a pertinent condition or disease described herein in a subject requires periodic (e.g., daily or twice daily) administration that continues for a period ranging from three days to two weeks or longer. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every day, every other day, or, if higher dose ranges are employed and tolerated by the subject, twice a week.

A dose can be formulated in animal models to achieve a concentration range that includes the IC₅₀ (the concentration of the agent that achieves a half-maximal inhibition of symptoms) as determined in cell culture. Such information can be used to more accurately determine useful doses in humans. Levels in stool or an enteric tissue sample can be measured, for example, by high performance liquid chromatography (HPLC). In general, the dose equivalent of the active ingredient of the composition of the invention is from about 1 ng/kg to about 1000 mg/kg, e.g., about 1 mg/kg to about 100 mg/kg for a typical subject.

The dosage of a composition of the present invention can be monitored and adjusted throughout treatment, depending on severity of symptoms, frequency of recurrence, and/or the physiological response to the therapeutic regimen. Those of skill in the art commonly engage in such adjustments in therapeutic regimens.

E. Production of the Native SERPIN B1 and its Variants

Optionally, the present disclosure provides coding sequence of the native SERPIN B1 or SERPIN B1 variant polypeptides have been engineered to match the codon usage pattern of the host (e.g., yeast) to maximize expression efficiency. Methods for codon optimization are readily available, for example, optimizer, accessible free of charge at genomes.urv.es/OPTIMIZER, OPTIMUMGENE™ algorithm from GenScript (Piscataway, N.J.), and GENEGPS® Expression Optimization Technology from DNA 2.0 (Newark, Calif.). In one embodiment, the coding sequence is codon-optimized for expression in S. cerevisiae. In some embodiments, the coding sequence is not codon-optimized.

The coding sequences of the SERPIN or its variants can be cloned into an expression vector, such as a plasmid, a cosmid, a phage, a virus (e.g., a plant virus), a bacterial artificial chromosome (BAC), a yeast artificial chromosome (YAC), or the like, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. In some embodiments, the expression vector is a Yeast episomal expression plasmid (YEp) containing a selectable marker.

In some embodiments, the promoter is a yeast promoter, e.g., the yeast ADH2 promoter. In other embodiments, the vector is an engineered yeast 2 micron plasmid.

Expression vectors comprising the coding sequences disclosed above can be transformed into a variety of host species or strains. In one embodiment, the host species is S. cerevisiae. In another embodiment, the S. cerevisiae is a strain that has been genetically modified to be protease-deficient.

Also provided herein is a method and composition of a fusion protein comprising the native SERPIN B1 or SERPIN B1 variant polypeptide and a second polypeptide. In some embodiments, the second polypeptide increases the half life of the fusion protein. For example, the second polypeptide may comprise or consist of a Fc portion of IgG, a single chain variable fragment (scFv), or an antibody.

In some embodiments, the native SERPIN B1 or SERPIN B1 variant polypeptide is modified post translationally, e.g., by pegylation.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1: Experimental Procedures

Materials—Purified human neutrophil elastase (HNE) and cathepsin G (CatG) were purchased from Lee Biosolutions (St. Louis, Mo.). Porcine pancreatic elastase (PPE), TLCK-treated bovine a chymotrypsin (BC), N-chlorosuccinimide (NCS), the rabbit polyclonal anti-Serpin B1 antibody (PN #SAB1101121) and a goat anti-rabbit HRP conjugate were purchased from Sigma-Aldrich (St. Louis, Mo.). The elastase substrate N-Succ-AAPV-pNA and BC substrate Succ-AAPF-pNA were purchased from Bachem Americas (Torrance, Calif.). 2-Mercaptoethanol (2-ME) was purchased from MP Biomedical (Santa Ana, Calif.). Sodium thiosulphate pentahydrate was purchased from VWR International (Visalia, Calif.). Chromatography Resins QXL fast-flow, Sephacryl S100 HR and Hitrap Q HP were purchased from GE Healthcare Life Sciences (Pittsburgh, Pa.). Hydroxyapatite (Macro-Prep Ceramic, Type 1, 40 um) was purchased from Biorad Laboratories (Hercules, Calif.). The Zorbax 300SB-C3 HPLC column was purchased from Agilent Technologies (Santa Clara, Calif.).

Expression of Recombinant Human sB1's in Yeast—Recombinant human serpin B1 (rhsB1) was expressed in and purified from the yeast S. cerevisiae. The human mRNA sequence encoding wild type human serpin B1 under the control of the yeast ADH2 promoter was synthesized and subcloned into the E. coli vector pUC57 at Genscript USA (New Jersey). The vector (2 ug) was digested with endonucleases and the appropriately sized DNA fragment recovered using a Zymoclean gel DNA recovery kit (Zymo Research, San Diego). This fragment was ligated into a yeast 2 um plasmid (pSB100) containing a ura- selectable marker and transformed into S. cerevisiae. Several colonies that grew on Ura-/8% glucose plates were screened for rhB1 expression by growing overnight at 30° C. in an inoculum of synthetic defined (SD) media (Sunrise Sciences, San Diego) lacking uracil but containing 8% glucose then transferring into yeast extract/peptone/2% glucose (YEPD) and growing for an additional 72 hours. Samples were taken at 24, 48 and 72 hours and analyzed for growth (OD₆₀₀), protein expression (SDS-PAGE) and rhB1 activity (inhibition of porcine pancreatic elastase). Several clones displaying good expression and activity of rhsB1 were selected and glycerol stocks prepared and frozen at −80° C. The Cys-344→serine mutation was introduced into the human serpin B1 coding sequence in pUC57 by site directed mutagenesis at Genscript USA (New Jersey) substituting C for G1031. This variant sequence was excised, subcloned, and transformed into yeast as described for the wild type rhsB1 protein. The coding sequences of the expression constructs were verified by DNA sequencing.

Purification of Recombinant Human sB1—rhsB1 was purified from yeast paste (50-100 g) using a combination of column chromatography and ammonium sulfate precipitation. As we were interested in the reactivity of the two cysteine residues in sB1 (Cys-214, Cys-344) and how they would behave once liberated from the highly reductive intracellular yeast environment, reducing agents were excluded from all purification steps. In brief, cells were subjected to glass bead lysis in a 10 mM tris buffer, pH 8.0 containing 1 mM EDTA (TE) using a bead beater (BioSpec Products, OK). Lysates were clarified by centrifugation at 20,000×g and the pH adjusted to 8.0 before loading directly onto an anion exchange column containing QXL fast flow resin equilibrated in TE buffer. Bound proteins were eluted with a 5CV gradient to 1M NaCl in the equilibration buffer. Fractions containing active rhsB1 were located using SDS-PAGE and a PPE inhibition assay (see: enzyme inhibition assays section), pooled and further purified and concentrated by consecutive 45% and 65% ammonium sulfate (AS) precipitation steps. For each of these steps solid AS was added to the rhsB1 containing pool, mixed at room temperature for 30 minutes then centrifuged for 20 minutes at 20,000×g. The 65% AS pellet was re-dissolved in a minimal volume of 10× TE buffer, pH 8.0 and loaded onto a size exclusion column containing Sephacryl 5100 HR resin equilibrated in TE, 100 mM NaCl, pH 7.4. Proteins were eluted at 5 mL/min and the peak containing monomeric rhB1 located by SDS-PAGE analysis. Peak fractions were pooled, dialyzed overnight into 10 mM NaCl, 5 mM sodium phosphate, pH 6.9 and loaded onto a column of ceramic hydroxyapatite (CHA) equilibrated in the dialysis buffer at 5 mL/min. The column was washed with equilibration buffer and bound proteins eluted with a 20 column volume gradient to 10 mM NaCl, 0.3M sodium phosphate, pH 6.9. Peaks eluting from the column were tested for rhsB1 content by SDS-PAGE and PPE inhibition. Fractions containing rhsB1 were pooled and concentrated on Vivaspin 20 ultrafiltration spin columns (Sartorius, Germany). Protein concentration was determined using the published extinction coefficient of 1.16 for a 1 mg/mL solution of rhsB1 and aliquots frozen at −80° C. (12).

Sensitivity of rhsB1 in Yeast Lysates to oxidation with N-chlorosuccinimide (NCS)—100 mg samples of yeast paste expressing rhsB1 were lysed by mechanical disruption. To a microfuge tube containing yeast paste, 0.75 mL of glass beads (0.5 mm) and 0.75 mL of TE (pH 8.0) were added and the tube mixed on a mini-vortexer (VWR scientific) for 3 pulses of 1 min each with 1 min rests on ice between pulses. Lysates containing soluble rhsB1 were clarified by centrifugation at 20,000×g for 10 min. Aliquots of clarified lysate (90 uL) were made to various concentrations of NCS (0-1 mM) by adding 10 uL of a 10× NCS concentrate (in TE, pH 8.0) to each aliquot and incubating at 30° C. for either 1, 5 or 30 minutes. Reactions were stopped by adding an excess of 1.0M sodium thiosulphate. Samples were taken and assayed for inhibition of porcine pancreatic elastase (PPE) and bovine chymotrypsin (BC) in PBS and analyzed by SDS-PAGE and western blotting for rhsB1 molecular form.

### Enzyme Inhibition Assays:

Porcine Pancreatic Elastase (PPE) assay—Inhibition of PPE was used both as a tool to detect rhsB1 containing fractions during purification and as an assay to assess the relative PPE inhibitory activity of PTM forms of rhsB1 present in oxidized yeast lysates and highly purified protein preparations. To monitor the purification steps, varying amounts of selected chromatographic peak fractions were incubated with PPE (100 or 200 nM) for up to 5 minutes in a final volume of 195 uL of PBS, pH 7.4 in a microtiter plate with or without 2-ME. The elastase substrate Succ-AAPV-pNA was then added to a final concentration of 1 mM and the release of free para-nitroanilide (pNA) monitored for several minutes at 405 nm on a plate reader (SPECTRAmax 340PC, Molecular Devices). Fractions containing rhsB1 were noted as those that fully inhibited PPE and were pooled and further processed as described in the purification section. When used as an assay to assess the relative inhibitory activity of rhsB1 proteins present in oxidized yeast lysates and highly purified preparations, a fixed concentration of PPE was used (100 nm). PPE was incubated with varying volumes of each sample in a fixed volume of PBS, pH 7.4 (195 uL) in a microtiter plate with or without 2-ME for varying time periods. Residual PPE activity was measured as described above. Similar assays can be performed assess the inhibitory activity of SERPIN B1 variants on a human pancreatic elastase.

Human Neutrophil Elastase (HNE) Assay—Human neutrophil elastase (HNE; 170 nM) was incubated with varying volumes of oxidized or non-oxidized yeast lysates and varying amounts of purified rhsB1 protein preparations in a fixed volume (195 uL) of PBS, pH 7.4 in a microtiter plate with or without 2-ME for varying time periods. Residual HNE activity was determined by adding the elastase substrate Succ-AAPV-pNA and monitoring as described above.

Bovine Chymotrypsin Assay—Bovine chymotrypsin (BC; 100 nM) was incubated with varying volumes of oxidized or non-oxidized yeast lysates and varying amounts of purified rhsB1 protein preparations in a fixed volume (195 uL) of PBS, pH 7.4 in a microtiter plate with or without 2-ME for varying time periods. Residual BC activity was determined by adding the BC substrate Succ-AAPF-pNA and monitoring as described above.

Human neutrophil Cathepsin G (CatG) Assay—Human neutrophil cathepsin G (CatG; 100 nM) was incubated with varying amounts of purified rhsB1 protein preparations in a fixed volume (195 uL) of PBS, pH 7.4 in a microtiter plate with or without 2-ME for varying time periods. Residual CatG activity was determined by adding the substrate Succ-AAPF-pNA and monitoring as described above for BC

Interaction of PPE, HNE, BC and CatG with rhsB1 proteins—Enzyme:rhsB1 complexes were visualized by SDS-PAGE and staining with coomassie blue G250. Typically, a fixed amount of enzyme was incubated with varying amounts of purified rhsB1 protein or a fixed amount of purified rhsB1 protein was incubated with varying amounts of enzyme for varying time periods sufficient to achieve either maximal complex formation or maximal conversion of rhsB1 proteins to lower molecular weight forms (based on the published association rate constants (4) and the data obtained from the enzyme assays described above). Residual enzyme was inhibited by the addition of a synthetic low molecular weight protease inhibitor cocktail (Sigma) and samples analyzed by SDS-PAGE.

Protein Sequence analysis of cleaved rhsB1M* and rhsB1D—Purified rhsB1M* and rhsB1D cleaved by PPE were further purified by ion exchange chromatography on a Hitrap Q HP column (5 mL) to remove PPE and residual low molecular weight synthetic protease inhibitors. Samples of each were sequenced through 7 cycles by Edman degradation at the UC Davis Molecular Structure Analysis Facility (Davis, Calif.).

### EXAMPLE 2: Purification in the Absence of Reducing Agents Yields Post-Translationally Modified Forms of rhsB1 that Lack Elastase Inhibitory Activity

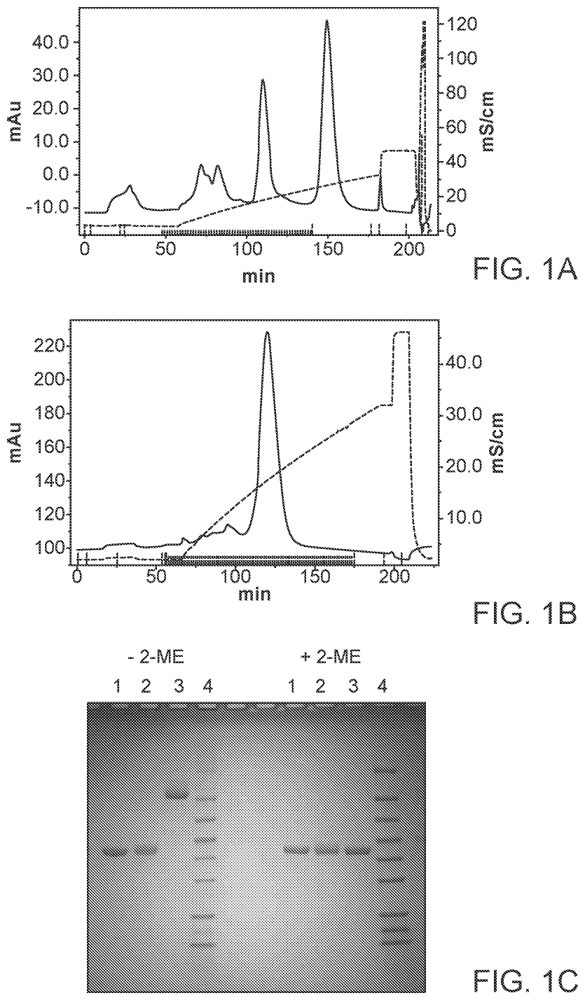

5 uL of yeast lysate containing rhsB1 were incubated and oxidized with varying concentrations of NCS. FIG. 2A. Data points are means of triplicate analyses +/−SE. Yeast expressing rhsB1 displayed a major single protein band visible on SDS-PAGE of ˜42 kDa molecular weight that specifically cross-reacts with anti-sB1 antisera in Western blotting (FIG. 2B, lane 1). Aliquots of yeast cell lysates expressing rhsB1 were active as inhibitors of PPE and BC in the absence of exogenously added reducing agents (FIGS. 2C and D). Thus, we initially attempted to purify rhsB1 using the method of Cooley et al (12) which employs 2-ME as the reducing agent of choice to preserve inhibitory activity but found the resultant product to be relatively impure as judged by RP-HPLC (<90%) and in some cases inactive due to limited proteolysis within the RSL (data not shown). We therefore modified the method to exclude reducing agents and incorporated 2 additional steps—ammonium sulfate fractionation and chromatography on a ceramic hydroxyapatite (CHA) resin. We found ammonium sulfate fractionation on the QXL FF pool to be a rapid way to further purify and concentrate rhsB1 prior to size exclusion chromatography. However, the product eluting from the size exclusion column was still relatively impure so CHA chromatography was incorporated as a final polishing step. Despite the loss of some product on size exclusion chromatography due to in-process dimerization of rhsB1, we were able to separate 2 rhsB1 containing peaks by CHA chromatography (FIG. 1A). The first major peak contained a monomeric form of rhsB1 of slightly higher molecular weight than fully reduced rhsB1 (FIG. 1C, lanes 2, 3 [+2-ME]) and the Ser-344 variant (FIG. 1C, lanes 1, 2 [−2-ME]). We refer to this species as rhsB1M*. The second peak contained a much higher molecular weight species of ˜84 kDa that could be reduced to the size of monomeric rhsB1 upon addition of 2-ME (FIG. 1C, lane(s) 3 [−/+2-ME]). This size is consistent with that of a dimerized form of rhsB1 hence we refer to this species as rhsB1D. The fact that we could not fully reduce rhsB1M* and rhsB1D to the same size with 2-ME was the first indication that the nature of each respective post translational modification is different and not just simple disulfide bond formation with another reduced thiol group (R-SH) containing molecule. In addition, during the purification we noted a gradual loss of EIA, along with an increase in the amount of rhsB1D, but no loss of CIA suggesting that once removed from endogenous yeast anti-oxidants such as glutathione and L-cysteine, the reactive site Cys-344 may become susceptible to post-translational modifications (PTM's). In support of this hypothesis, purification of the Ser-344 variant on CHA chromatography yielded only a single monomeric rhsB1 containing peak eluting at a similar position to rhsB1M* (FIG. 1B). Furthermore, the Ser-344 variant protein was not of a higher molecular weight than that present in the lysate (data not shown) and its mobility in SDS-PAGE gels was unaffected by 2-ME (FIG. 1C, lane(s) 1). These results indicate that PTM's at Cys-344 are most likely responsible for the molecular weight shifts occurring in rhsB1M* and rhsB1D rather than modifications at Cys-214 or other residues within rhsB1.

### Example 3. Direct Oxidation of rhsB1 in Yeast Lysates Rapidly Induces a PTM Similar to that Observed in rhsB1M*

rhsB1 in yeast lysates was sensitive to oxidation with NCS (as determined by the loss of EIA) which induced a molecular weight shift similar to that seen in rhsB1M*. Aliquots (5 uL) of untreated lysates were able to completely inhibit PPE (100 nM) activity but this EIA was rapidly and specifically lost in a dose dependent manner upon oxidation with NCS (FIGS. 2A, C). NCS concentrations >600 μM were able to diminish rhsB1's EIA and concomitantly induced the appearance of a specific band of slightly higher molecular weight than non-oxidized rhsB1 (FIG. 2B, lane 2 [SDS-PAGE and Western blot]). We observed this reaction to be rapid and nearly complete within the earliest time point (1 min) that we tested. No other major yeast protein visible on SDS-PAGE underwent this shift. In contrast, oxidized lysates containing rhsB1 retained their CIA (FIG. 2D).

### Example 4. rhsB1D and rhsB1M* are Substrates for Porcine Pancreatic Elastase

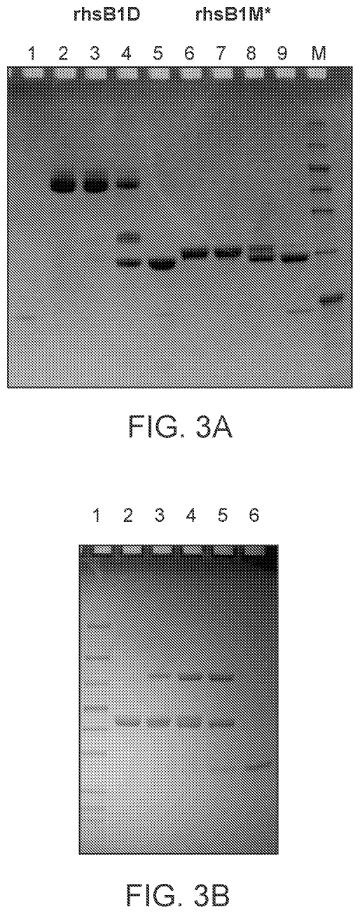

In the absence of 2-ME neither rhsB1M* nor rhsB1D were able to inhibit PPE. Instead, both were readily cleaved to lower molecular weight species. FIG. 3A (lanes 3-5 and 7-9) shows the cleavage profiles of rhsB1D and rhsB1M* respectively, generated by enzyme to inhibitor (E:I) molar ratios ranging from 1:10 to 1:1,000. Both rhsB1D and rhsB1M* were specifically degraded to lower molecular species of approximately 38 kDa at E:I molar ratios ≥1:100 within a 30 minute time period. For serpins this degradation pattern is usually indicative of limited proteolysis within the RSL especially when accompanied by the specific loss of an enzyme inhibitory function ascribed to that serpin. In the case of rhsB1D, an intermediate degradation species of ˜46 kDa (FIG. 3A, lane 4) and a transiently staining one of ˜8-10 kDa were also observed (lane 5). The ˜46 kDa species size is consistent with that of an intact rhsB1 monomer (˜42 kDa) disulfide bonded via Cys-344 to an RSL peptide (˜4 kDa) released from a cleaved rhsB1 molecule. The 8-10 kDa species likely comprises two RSL peptides disulfide-bonded together via Cys-344.

In the presence of 25 mM 2-ME, rhsB1M* was still unable to efficiently inhibit PPE and was also degraded to the lower molecular species as described above (data not shown). In contrast, rhsB1D was reduced by 25 mM 2-ME to an active monomeric rhsB1 able to form SDS-stable complexes with PPE in a dose dependent manner (FIG. 3B). However, this gel clearly shows that in addition to the higher molecular weight E:I complexes formed another lower molecular species similar to the cleaved forms described above was generated at E:I molar ratios >0.25 (˜38 kDa).

### Example 5. rhsB1D and rhsB1M* are Cleaved at Multiple Sites within the Reactive Site Loop by Porcine Pancreatic Elastase

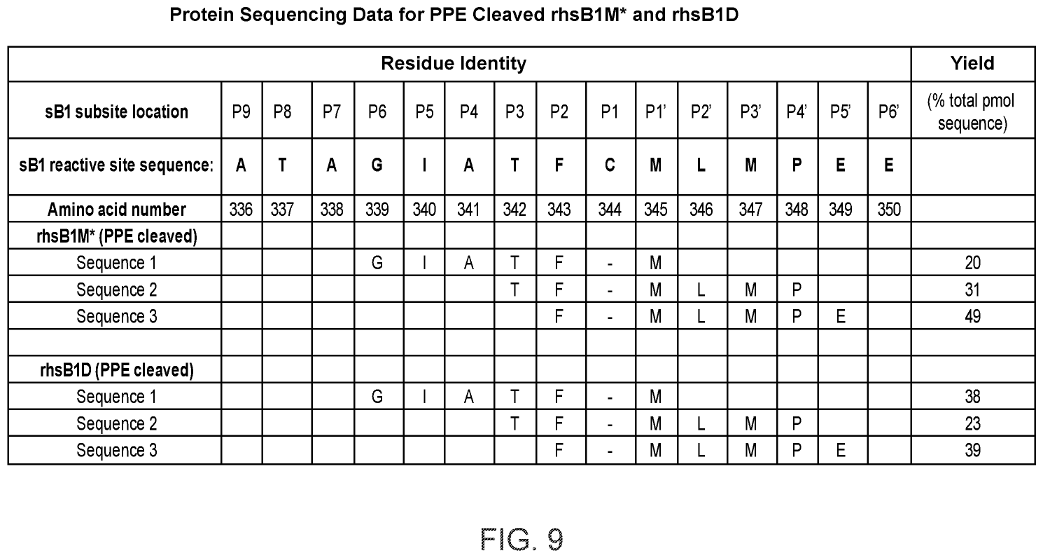

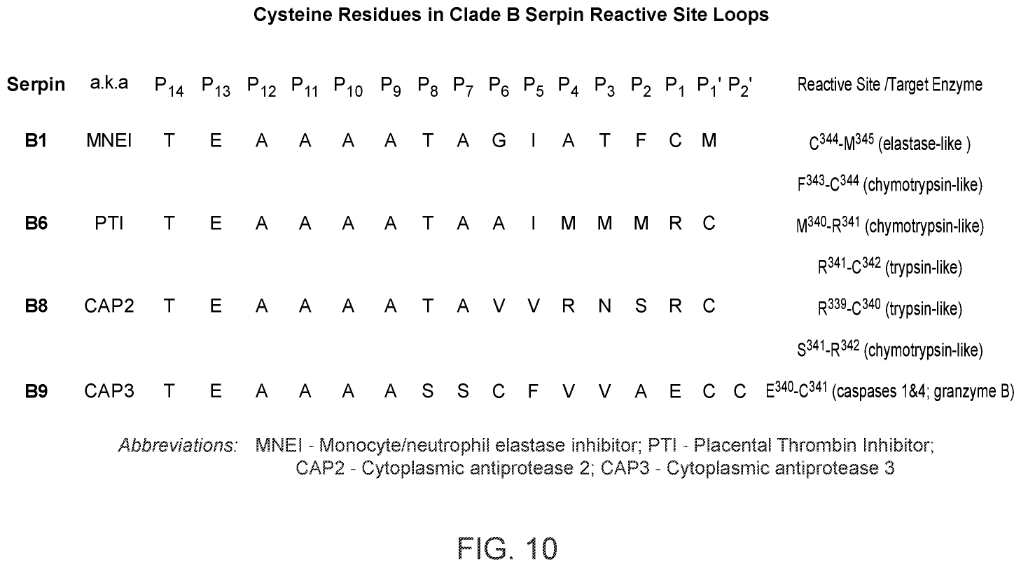

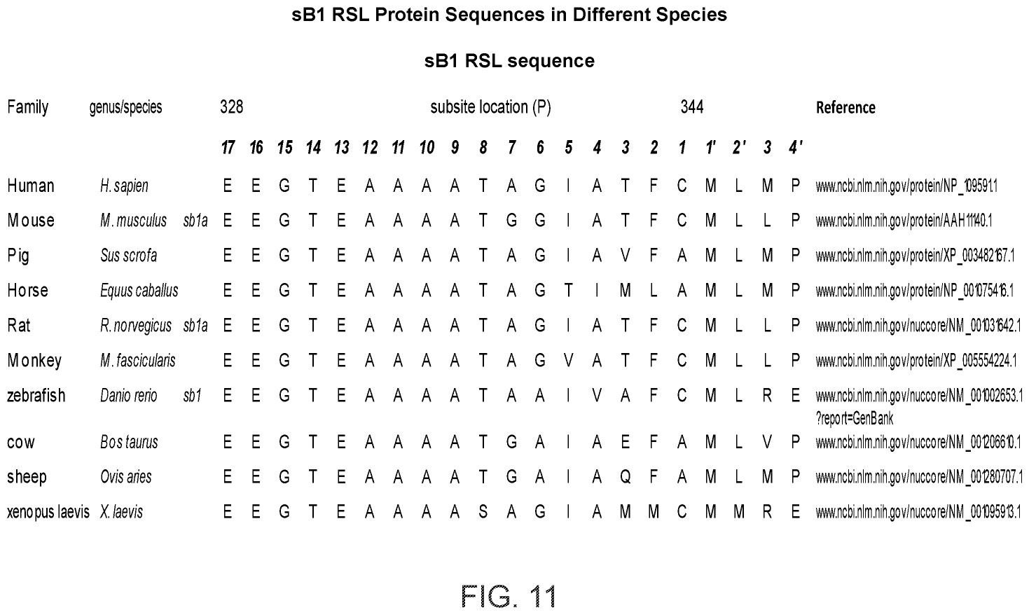

Direct protein sequence analysis of rhsB1M* cleaved by PPE yielded 3 separate but overlapping sequences derived from the RSL of sB1 (Table 1, FIG. 9), a resultant consistent with the 3 peaks observed on RP-HPLC. The major sequence present (˜49%) was derived by cleavage of the Thr-342:Phe-343 bond. Two (2) other sequences were present in lower amounts and were derived by cleavage of the Ala-338:Gly-339 (˜20%) and Ala-341:Thr-342 (˜31%) bonds. Sequence analysis of PPE cleaved rhsB1D yielded the exact same 3 sequences but the percentage yield of each was different. The amount of cleavage of the Thr-342:Phe-343 and Ala-341:Thr-342 bonds decreased to 39% and 23% respectively while cleavage at the Ala-338:Gly-339 bond increased to 38%. These results are consistent with the reported specificity of PPE and confirm Cys-344 as the residue responsible for disulfide bond formation in rhsB1D. Cys-344 is the only cysteine residue present in the RSL that could partake in this interaction and is C-terminal to the elastase cleavage sites whereas Cys-214 is part of the 38 kDa species.

No protein sequence data was obtained from the N-terminal amino acids of rhsB1 most likely due to acetylation of the N-terminal methionine's α-amino group by N-acetyltransferase B (NatB) interfering with the Edman degradation process chemistry. This has been observed for other N-acetylated clade B serpins and proteins expressed intracellularly (29).

### Example 6. Reduction of rhsB1D to Restore Maximal Elastase Inhibitory Activity Requires High Concentrations of 2-ME

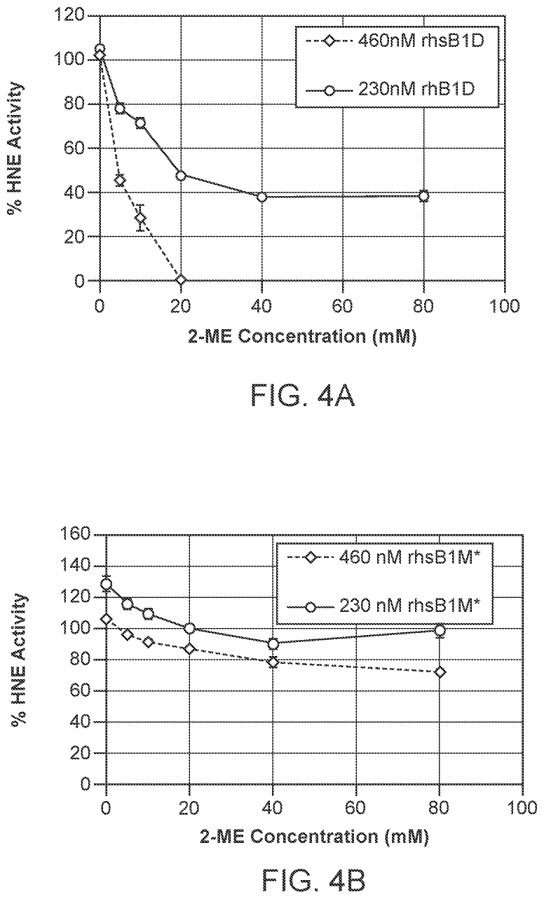

To accurately evaluate the inhibitory activity of fully reduced rhsB1D or rhsB1M* on human neutrophil elastase (HNE) we titrated a fixed amount of each (230 or 460 nM) with differing concentrations of 2-ME and then reacted them with a fixed amount of HNE (170 nM). Appropriate controls evaluating the effect of 2-ME alone on HNE activity were included. FIG. 4A shows that increasing the concentration of 2-ME up to 40 mM in solutions containing 230 nM rhsB1D resulted in a progressive decrease in the residual activity of HNE to ˜40% of the initial value. Concentrations of 2-ME higher than this (up to 0.5 M) had no further impact while doubling the amount of rhsB1D in the assay resulted in complete inhibition of HNE. This indicates that the Cys-344:Cys-344 intermolecular disulfide bond in rhsB1D requires relatively high concentrations of a reducing agent—in the case of 2-ME, at least ≥25 mM—to fully restore EIA.

In contrast, titration of 230 nM rhsB1M* with 2-ME only had a small effect on EIA at any of the concentrations tested (FIG. 4B). Nevertheless, at 460 nM, this effect was slightly enhanced suggesting that a small portion of the rhsB1M* preparation is susceptible to reduction and has not been irreversibly modified.

### Example 7. rhsB1D and rhsB1M* are Also Substrates for HNE

The interaction of rhsB1D and rhsB1M* with HNE provided results very similar to those described above for PPE. Neither were able to significantly inhibit HNE in the absence of a reducing agent and both displayed degradation patterns similar to those seen in FIG. 3A (FIGS. 5B and 5D; PBS). FIGS. 5A and B show the interaction of a fixed concentration of HNE with increasing amounts of rhsB1D in the presence or absence of 40 mM 2-ME. The molar amount of an active serpin required to fully inhibit an equimolar amount of an active enzyme is often referred to as the stoichiometry of inhibition or SOI (30) and even though we have not determined the specific activity of our enzyme preparations it is clear from the kinetic data in FIG. 5A that the SOI of fully reduced rhsB1D with HNE is ˜2.1. This data confirms that the reduced form of rhsB1D is absolutely required for the inhibition of HNE as in the absence of 2-ME no complexes were seen and the dimer was fully cleaved to yield RSL-cleaved rhsB1 (FIG. 5B; “PBS”) whereas in the presence of 2-ME, HNE:rhsB1 complexes were readily observed at −68 kDa with an intermediate species at ˜57 kDa and RSL-cleaved rhsB1 at ˜42 kDa (FIG. 5B, PBS+40 mM 2-ME). The intermediate species was not seen when HNE inhibition was maximal and only complex, RSL-cleaved and intact rhsB1 were visible. The intermediate species may be produced by the action of free enzyme on the complex.

In contrast, the SOI of reduced rhsB1M* with HNE is ˜13 (data not shown) with the majority of rhsB1M* being catalytically cleaved within the RSL and a small amount of intermediate ˜57 kDa complex visible on SDS-PAGE (FIG. 5C, D; PBS+40 mM 2-ME). This provides further evidence that a fraction of rhsB1M* in this preparation was not irreversibly oxidized.

### Example 8. rhsB1D and rhsB1M* Retain Chymotrypsin Inhibitory Activity but with Altered Stoichiometries of Inhibition

In contrast to the elastases, BC was inhibited by both rhsB1D and rhsB1M* in the presence or absence of 2-ME although their SOI's were altered. Our kinetic data indicated that in the presence of 2-ME, rhsB1D had an SOI approaching 0.5 (FIG. 6A). However, such a low SOI is theoretically impossible and likely reflects a specific activity of ≤50% for the BC preparation. In the absence of 2-ME this ratio increased to ˜1.0 and did not change when samples were incubated for extended time periods of up to 1 hr—indicating that the reaction had gone to completion. One explanation for this doubling in SOI in the absence of 2-ME may be that once BC is bound to one of the RSL's in the intact dimer access to the other RSL may be sterically hindered thus allowing inhibition of only one BC molecule by one dimer molecule. Alternatively, the structural changes accompanying complex formation may render the second RSL unable to effectively interact with BC to form complexes. SDS-PAGE data corroborated the kinetic data and showed the disappearance of a fixed concentration of free BC upon titration with increasing amounts of rhsB1D in the presence or absence of 2-ME (FIG. 6B). In the absence of 2-ME (PBS), rather than forming a complex of predicted molecular weight ˜109 kDa consisting of the dimer and the enzyme, the dimer appeared to dissociate concomitant with the formation of a lower molecular weight band of ˜67 kDa and trace amounts of a band at ˜42 kDa. The former band has a molecular weight consistent with that of the complex formed between non-reduced BC (˜25 kDa, PBS, E) and monomeric rhsB1 (˜42 kDa, PBS+40 mM 2-ME, I); the latter with that of either intact or RSL-cleaved rhsB1. In the presence of 2-ME, BC dissociates into two chains of ˜15 and ˜10 kDa the smaller of which retains the active site Ser-195. Thus much smaller complexes and complex intermediates (˜52 and ˜48 kDa) are formed between rhsB1 and BC under these conditions in addition to a small amount of RSL-cleaved rhsB1 (PBS+40 mM 2-ME).

The SOI for rhsB1M* with BC was ˜3 in the absence or presence of 2-ME with a trend to a slightly lower SOI in the presence of 2-ME (FIG. 6C, D). On SDS-PAGE the molecular weights of the complexes formed with BC in the absence of 2-ME were similar to those seen with rhsB1D in the absence of 2-ME except more RSL-cleaved rhsB1M* was visually apparent—a finding consistent with the higher observed SOI.

### Example 9. The Interaction of rhsB1D and rhsB1M* with CatG was Similar to that with BC

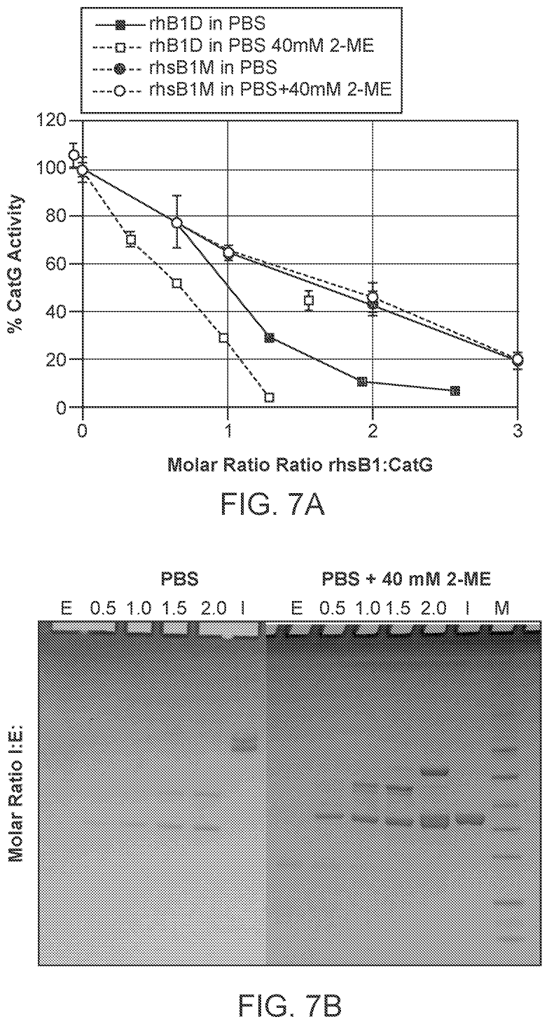

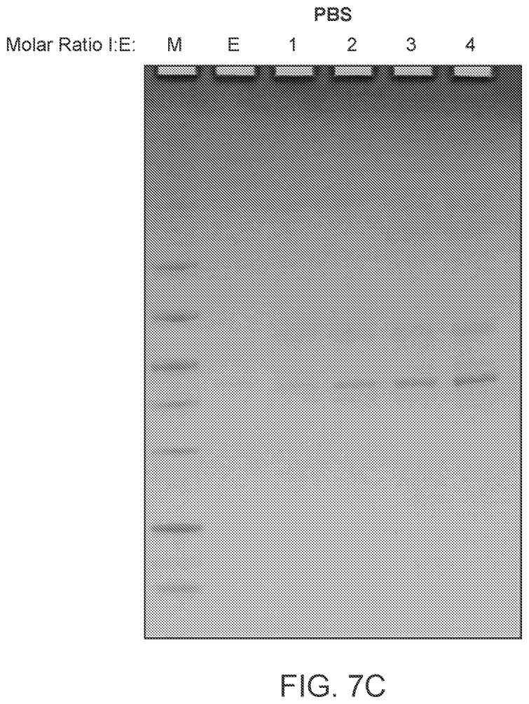

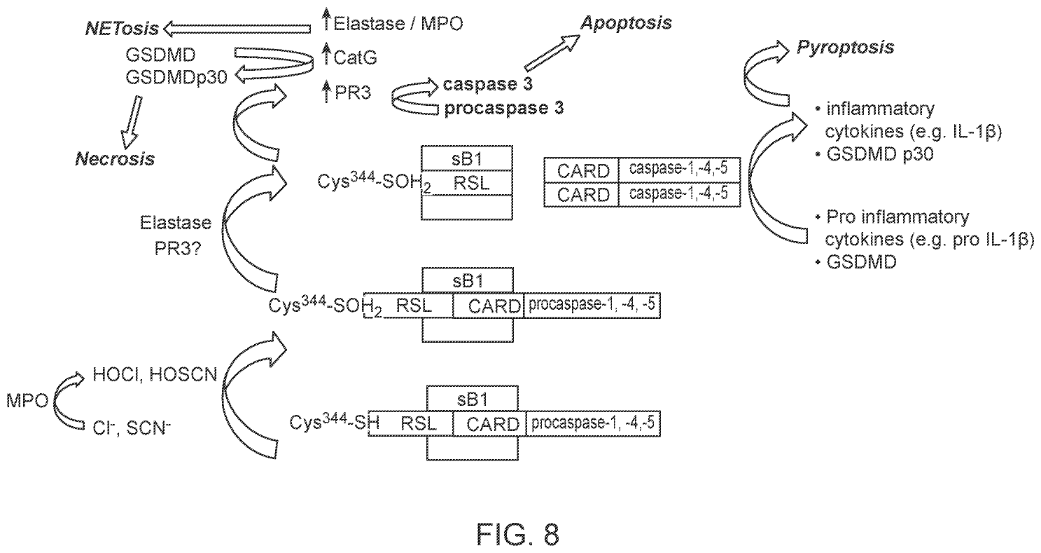

As CatG is closely related to chymotrypsin we anticipated that its' interaction with rhsB1D and rhsB1M* would generate similar data to that described above for BC and rhsB1D and rhsB1M*. The results did show a similar interaction kinetically, and by SDS-PAGE analysis of the reaction products. The reduced dimer appeared twice as effective at inhibiting CatG when compared to the unreduced dimer although the SOI for each was approximately double that described for the interaction of rhB1D with BC. This likely reflects either a higher specific activity of the CatG preparation, a higher turnover of rhB1D by CatG during complex formation or, a combination of the two. In support of the second hypothesis, it would appear from the comparative SDS-PAGE profiles in FIGS. 6B and 7B that the complexes formed between BC and rhsB1D in the absence of 2-ME are much more stable than those formed between CatG and rhsB1D and yield very little complex intermediate or RSL-cleaved rhsB1. The comparative SDS-PAGE profiles for the interactions of rhsB1M* with either BC or CatG also demonstrate this comparative instability with more rhsB1M* partitioning into the RSL-cleavage pathway than forming either intermediate or full complexes when reacted with CatG than when reacted with BC (FIGS. 6D, 7C). Thus the SOI for the interaction of CatG with either rhsB1D or rhsB1M* is likely higher than that observed for the interaction of BC with rhsB1D or rhsB1M* respectively.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

### Example 10. Characterizing the Wild Type sB1

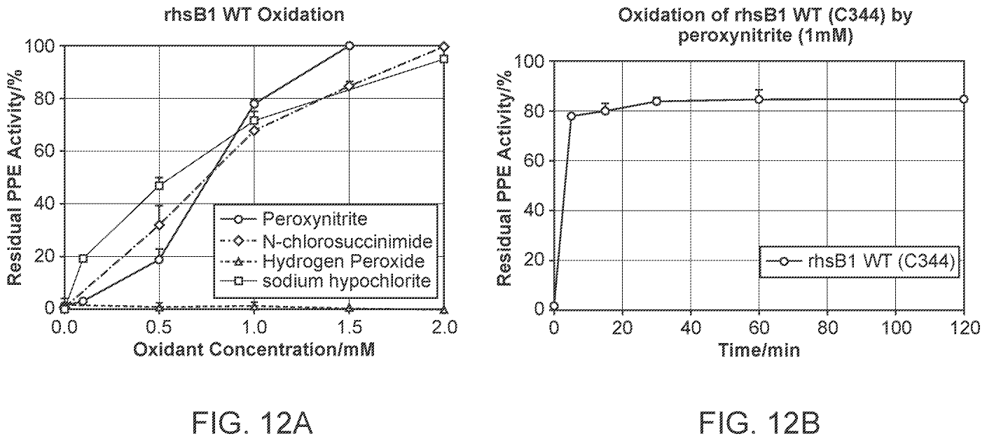

This experiment was conducted to analyze the effect of physiologically relevant ROS and RNS on the elastase inhibition activity of sB1. Yeast expressing wild-type serpin B1 (rhsB1 WT (C344) were lysed in PBS, 1 mM EDTA, pH 7.5 and the soluble lysate containing wild-type (C344) serpin B1 separated from insoluble cell debri by centrifugation. Enzyme (PPE) was titrated with the lysate until no PPE activity was observed. The conditions in this experiment were similar to those described in Example 1 and the rhsB1 was present as an unoxidized free monomer rhsB1 C344-SH stabilized by endogenous anti-oxidants present in the yeast lysate. Separate samples of the lysate were treated with varying concentrations of each ROS/RNS for varying times and the reaction stopped with an excess of sodium thiosulphate. Oxidized lysate samples were then reacted with PPE to re-assess inhibitory activity after adjusting for treatment related volume changes using the method described in the legend for FIG. 1. FIG. 12 Panel A shows the effect of different concentrations of peroxynitrite (both an ROS and an RNS), N-chlorosuccinimide (ROS), hydrogen peroxide (ROS) and sodium hypochlorite (ROS) on the elastase inhibitory activity present in each sample. FIG. 12 Panel B shows that a 1 mM concentration of peroxynitrite can rapidly inactivate the elastase inhibitory activity of wild-type serpin B1- at the earliest time-point measured (5 minutes) 80% of PPE activity was restored. These results show that the wild-type serpin B1 (C344) is rapidly inactivated as an elastase inhibitor by reactive oxygen and nitrogen species (ROS/RNS) that are strong oxidants (e.g., peroxynitrite) but not hydrogen peroxide (H₂O₂), also a ROS but a weaker oxidant. This difference is likely due to the C344-SH group in rhsB1 being stabilized by the surrounding amino acids in the protein, and it can only be oxidized by strong oxidants.

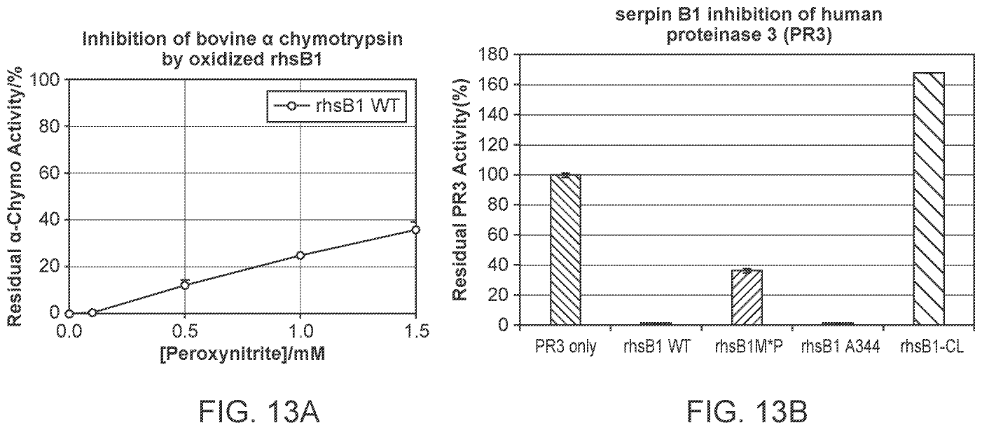

Next we performed an experiment to test the ability of oxidized wild-type sB1 to inhibit other proteases. Yeast lysate containing wild-type serpin B1 (C344) that had been treated and inactivated with peroxynitrite was used to assess its ability to inhibit bovine α-chymotrypsin (FIG. 13, panel A). The initial titration of unoxidized lysate against the enzyme to establish baseline conditions of maximal inhibition was carried out as described for PPE except using the chymotrypsin chromogenic substrate Succ-AAPF-pNA. The results show that oxidation of the wild type serpin B1 (C344) reduces its ability to inhibit bovine α-chymotrypsin.

We then purified peroxynitrite inactivated wild-type serpin B1 and tested its ability to inhibit neutrophil proteinase 3 (PR3; FIG. 13, Panel B). In this experiment 10 uL of 3.3 uM PR3 was mixed with a 2-fold molar excess of each purified inhibitor in a final volume of 195 uL (PBS, pH 7.4) in a microtiter plate. Samples were incubated 5 min then 5 uL of the chromogenic substrate methoxy Succ-AAPV-pNA added (final concentration ˜1 mM) and residual enzyme activity monitored at Δ405 nm for 20 min. The results show that wild-type rhsB1 (rhsB1-WT) can completely inhibit the chromogenic substrate cleaving activity of proteinase 3 (PR3) but peroxynitrite oxidized rhsB1 (rhsB1M*P) has lost ˜40% of its inhibitory activity (FIG. 13 panel B). In contrast, the oxidation resistant C344A variant (rhsB1 A344) did not lose any inhibitory activity. Finally, the elastase cleaved form of rhsB1 (rhsB1-CL) lost all inhibitory activity and actually stimulated PR3 activity by ˜50% confirming that cleavage within the reactive site loop completely destroys all protease inhibition activity.

### Example 11. Assessing the Elastase Inhibition Activity of the sB1 Variants

Serpin B1 reactive site variants were constructed by changing the DNA coding sequence for the wild-type amino acid (C344) by site-directed mutagenesis. Sequences were verified by DNA sequencing, cloned into a yeast expression vector and transformed into the yeast S. cerevisiae. Variant proteins were expressed and purified as previously described.

Human Alpha 1-Antitrypsin (AAT) was included as a reference control. The molecular weight of AAT is approx. 54,000 daltons compared to approx. 42,500 daltons for serpin B1.

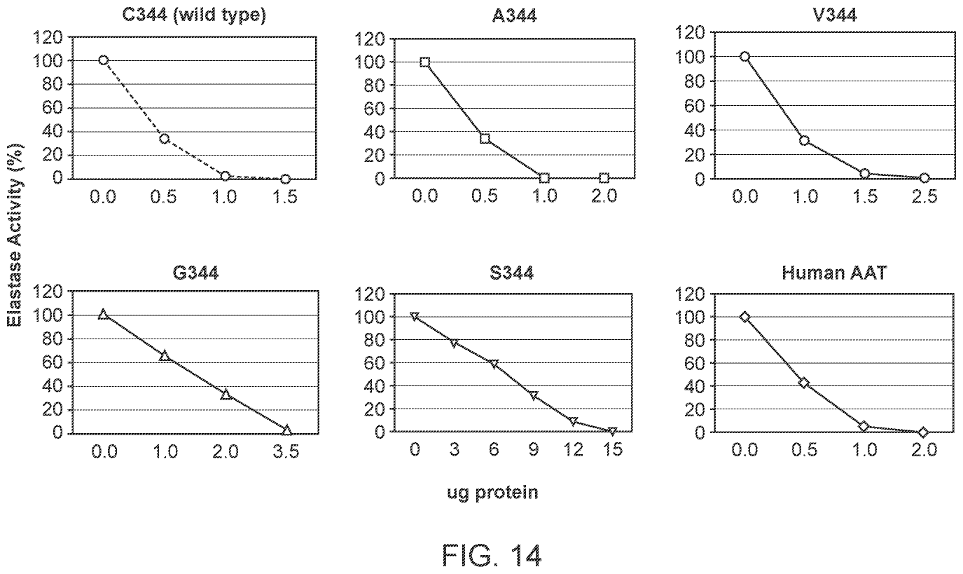

Enzyme assays: A concentration of porcine pancreatic elastase (PPE) was used that yielded a Vmax of ˜400 mAU/min at 405 nm with the chromogenic substrate Succ-AAA-pNA in a final volume of 200 uL in a microtiter plate. This was typically 160 nM. The order of addition of reagents to a microtiter plate well was as follows: 1. Enzyme (8-10 uL of an 0.1 mg/mL working stock of PPE); 2. Buffer (PBS, pH7.4, +40 mM 2-mercaptoethanol); 3. Inhibitor. The solution was mixed, incubated for 5 min at 25° C., then 5 uL of chromogenic substrate was added to a final concentration of 1 mM and enzyme activity recorded for 2 min on a SpectraMax 340 PC microtiter plate reader (Molecular Devices). Each assay was performed in triplicate on 2 occasions. Results showed that all four variants exhibit elastase inhibition activity but they do so with very different efficiencies (FIG. 14). Only the C344A (A344) variant required the same amount of purified protein (lug) to fully inhibit PPE as did wild-type rhsB1 (C344 (wild type)) and was equivalent to purified human AAT. The C344V (V344) variant was the next best, requiring 1.5 ug of purified protein while the C344G variant required 3.5 ug of protein to achieve the same level of inhibition. The C344S variant was a poor inhibitor of PPE, requiring 13 ug of purified protein to achieve complete inhibition. Ideally, the C344A variant is the substitution of choice for an oxidation resistant form of the protein followed by C334V and C344G then C344S. Clearly, making seemingly conservative substitutions for C344 in serpin B1 results in proteins that have very different abilities to inhibit PPE.

### C344S is a Poor Inhibitor of Elastase

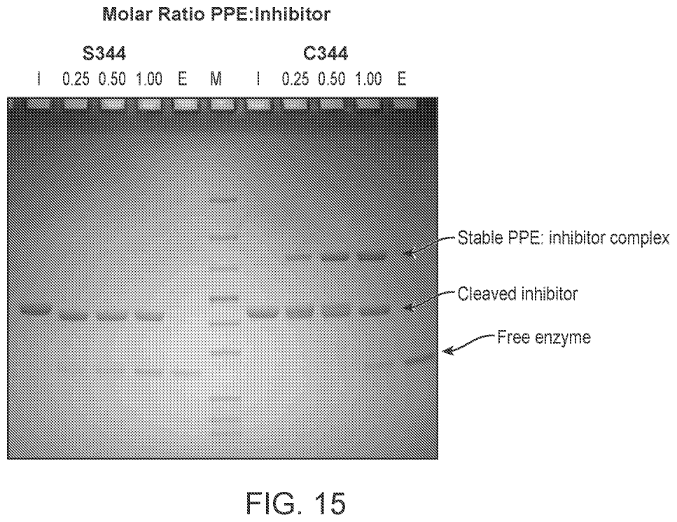

In this experiment a fixed amount of wild-type rhsB1 (C344) or the C344S variant (S344) were incubated with varying amounts of PPE at the molar ratios indicated. The reaction volume was fixed at 20 uL. PPE was added first followed by buffer and inhibitor. Samples were incubated for 5 min at 25° C. then a synthetic protease inhibitor mix (Sigma cat #P8215) added to quench active enzyme, SDS-PAGE loading buffer added and samples heated for 5 min. Reaction products were resolved by 4-20% SDS-PAGE and visualized by staining with coomassie blue R250 (FIG. 15). I represents inhibitor (serpin B1 protein); E represents porcine pancreatic elastase (PPE); M representas SDS-PAGE molecular weight markers (10, 15, 20, 30, 40, 50, 60, 80 and 120 kDa). The results show that the C344S serpin B1 variant is rapidly cleaved and inactivated by PPE at enzyme:inhibitor ratios that would normally form SDS and heat stable complexes with wild-type (C344) serpin B1. For those skilled in the art of protease inhibitor biology this type of interaction is referred to as the “stoichiometry of inhibition” (SI) where the serpin is either a very “efficient” inhibitor of the target enzyme and can form stable complexes at, or near, equimolar inhibitor:enzyme ratios in which the enzyme is inactive, or the serpin is a very poor inhibitor and most of it partitions into the substrate pathway where it gets catalytically cleaved—never forming stable complexes. This is very important physiologically as much larger amounts of poor inhibitors would be required to inhibit a target enzyme compared with an efficient inhibitor.

The C344A rhsB1 Variant is Resistant to Oxidation

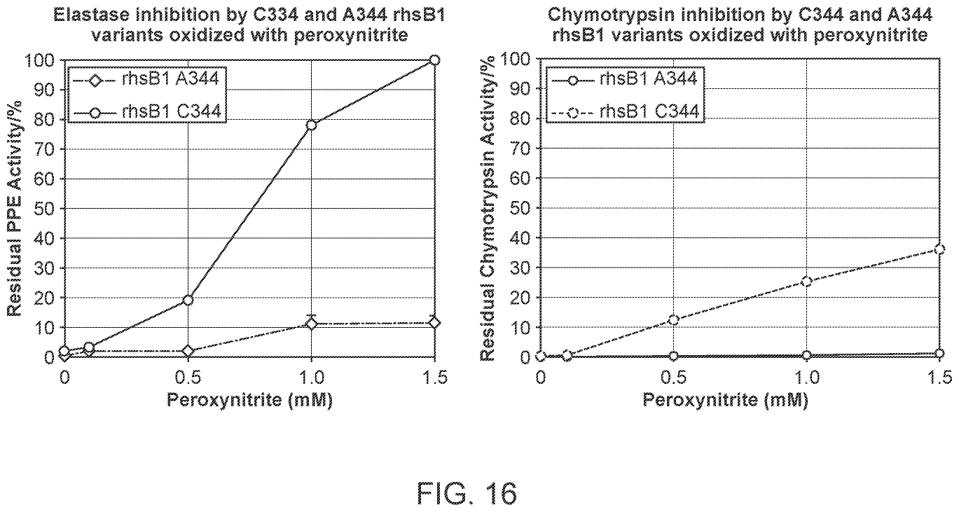

In this experiment yeast lysates containing either wild-type serpin B1 (C344) or the C344A variant were treated with the oxidant peroxynitrite as described previously. Samples were then tested for their ability to inhibit elastase and chymotrypsin as described in FIGS. 3 and 4. The results (shown in FIG. 16) show that the serpin B1 C344A variant retains elastase and chymotrypsin inhibitory activity in the presence of peroxynitrite while wild-type serpin B1 (rhsB1 C344) is rapidly inactivated by increasing amounts of peroxynitrite.

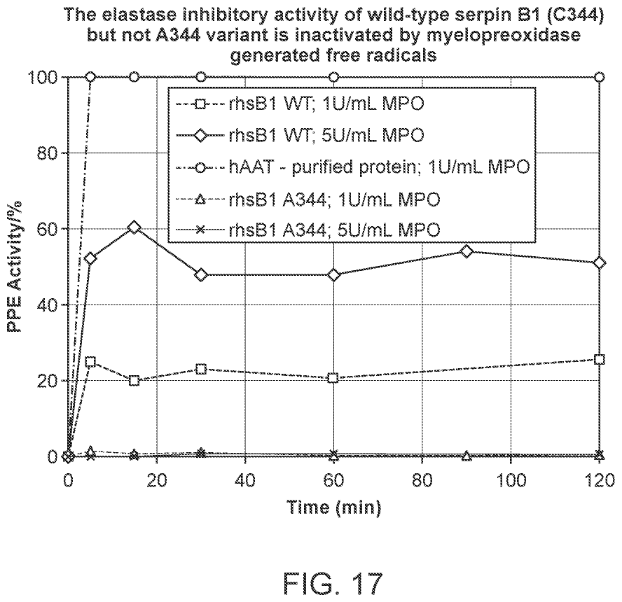

In this experiment yeast expressing either serpin B1 wild-type protein (rhsB1 WT) or the C344A variant (rhsB1 A344) were lysed in PBS, pH 7.5. Enzyme (PPE) was titrated with lysate(s) until no PPE activity was observed. Lysate(s) were then incubated with 1 or 5 U of purified human neutrophil myeloperoxidase (MPO)+25 mM sodium chloride and 80 uM H2O2 for varying times and the reaction stopped with an excess of sodium thiosulphate. Oxidized lysate samples were reacted with PPE to re-assess inhibitory activity as previously described. Purified human AAT (hAAT) was used as a control as it has previously been shown to be inactivated by MPO generated free radicals. The results (shown in FIG. 17) indicate that wild-type serpin B1 (rhsB1 WT) and hAAT, but not the C344A variant (rhsB1 A344) were rapidly inactivated by myeloperoxidase (MPO) generated free radicals in a dose dependent manner.

### REFERENCES

1. Remold-O'Donnell, E. (1985) A fast-acting elastase inhibitor in human monocytes. J. Exp. Med. 162, 2142-2155

2. Remold-O'Donnell, E., Nixon, J. C., and Rose R. M. (1989) Elastase inhibitor: Characterization of the human elastase inhibitor molecule associated with monocytes, macrophages and neutrophils. J Exp. Med. 169, 1071-1086
3. Remold-O'Donnell, E., Chin, J., and Alberts, M. (1992) Sequence and molecular characterization of human monocyte/neutrophil elastase inhibitor. Proc. Natl. Acad. Sci., USA 89, 5635-5639
4. Cooley, J., Takayama, T. K., Shapiro, S. D., Schecter, N. M., and Remold-O'Donnell, E. (2001) The serpin MNEI inhibits elastase-like and chymotrypsin-like serine proteases through efficient reactions at two active sites. Biochemistry 40, 15762-15770
5. Wang, L., Li, Q., Wu, L., Liu, S., Zhang, Y., Yang, X., Zhu, P., Zhang, H., Zhang, K., Lou, J., Liu, P., Tong, L., Sun, F., and Fan, Z. (2013) Identification of serpin B1 as a physiological inhibitor of human granzyme H. J. Immunol. 190, 1319-1330
6. Benerafa, C., Priebe, P. G., and Remold-O'Donnell, E. (2007) The neutrophil serine proteinase inhibitor serpinb 1 preserves lung defense functions in Pseudomonas aeruginosa infection. JEM204 (8), 1901-1909
7. Gong, D., Farley, K., White, M., Hartshorn, K. L., Benerafa, C., and Remold-O'Donnell, E. (2011) Critical role of serpinB1 in regulating inflammatory responses in pulmonary influenza infection. JID 204, 592-600
8. Benerafa, C., LeCuyer, T. E., Baumann, M., Stolley, J. M., Cremona, T. P., and Remold-O'Donnell, E. (2011). SerpinB1 protects the mature neutrophil reserve in the bone marrow. JLB 90, 21-29
9. Baumann M., Pham, C. T. N., and Benerafa, C. (2013) SerpinB1 is critical for neutrophil survival through cell autonomous inhibition of cathepsin G. Blood 121(19), 3900-3907
10. Farley, K., Stolley, J. M., Zhao, P., Cooley, J., and Remold-O'Donnell, E. (2012) A serpinB1 regulatory mechanism is essential for restricting neutrophil extracellular trap generation. J Immunol. 189, 4574-4581
11. Zhao, P., Hou, L., Farley, K., Sundrud, M. S., and Remold-O'Donnell, E. (2014) SerpinB1 regulates homeostatic expansion of IL-17⁺γδ and CD4⁺ Th17 cells. JLB 95, 521-530
12. Cooley, J., Mathieu, B., Remold-O'Donnell, E., and Mandle, R. J. (1998) Production of recombinant human monocyte/neutrophil elastase inhibitor (rM/NEI). Protein Expression and Purification 14, 38-44
13. Rees, D. D., Rogers, R. R., Cooley, J. A., Mandle, R. J., Kenney, D. M., and Remold-O'Donnell, E. (1999) Recombinant human monocyte/neutrophil elastase inhibitor protects rat lungs against injury from cystic fibrosis airway secretions. Am. J. Respir. Cell. Mol. Biol., 20, 69-78
14. Woods, D. E., Cantin, A., Cooley, J., Kenney, D. M., and Remold-O'Donnell, E. (2005) Aerosol treatment with MNEI suppresses bacterial proliferation in a model of chronic Pseudomonas aeruginosa lung infection. Pediatr. Pulmon. 39(2), 141-149
15. Torriglia, A., Perani, P., Brossas, J. Y., Chaudun, E., Treton, J., Courtois, Y., and Counis, M-F. (1998) L-DNase II, a molecule that links proteases and endonucleases in apoptosis, derives from the ubiquitous serpin leukocyte elastase inhibitor. Mol. Cell. Biol. 18(6), 3612-3619
16. Padron-Barthe, L., Lepretre, C., Martin, E., Counis, M-F., and Torriglia A. (2007) Conformational modification of serpins transforms leukocyte elastase inhibitor into an endonuclease involved in apoptosis. Mol. Cell. Biol. 27(11), 4028-4036
17. El Ouaamari, A., Dirice, E., Gedeon, N., Hu, J., Zhou, J-Y., Shirakawa, J., Hou, L., Goodman, J., Karampelias, C., Qiang, G., Boucher, J., Martinez, R., Gritsenko, M. A., De Jesus, D. F., Kahraman, S., Bhatt, S., Smith, R. D., Beer, H-D., Jungtrakoon, P., Gong, Y., Goldfine, A. B., Liew, C. W., Doria, A., Andersson, O., Qian, W-J., Remold-O'Donnell, E., and Kulkarni, R. N., (2016) SerpinB1 promotes pancreatic β-cell proliferation. Cell Metab. 23, 1-12
18. Choi, Y. J., Kim, S., Choi, Y., Nielsen, T. B., Yan, J., Lu, A., Ruan, J., Lee, H-R., Wu, H., Spellberg, B., and Jung, J. U. (2019) SERPINB1-mediated checkpoint of inflammatory caspase activation. Nat. Immunol. 20(3), 276-287
19. Yao, W., Li, H., Luo, G., Li, X., Chen, C., Yuan, D., Chi, X., Xia, Z. and Hei, Z. (2017) SERPINB1 ameliorates acute lung injury in liver transplantation through ERK1/2-mediated STAT3-dependent HO-1 induction. Free Radic. Biol. Med. 108, 542-553.
20. Fitzpatrick, P. A., Wong, D. T., Barr, P. J. and Pemberton, P. A. (1996) Functional implications of the modeled structure of maspin. Protein Eng. 9(7), 585-589.
21. Mahajan, N., Shi, H. Y. Lukas, T. J. and Zhang, M. (2013) Tumor suppressive maspin functions as a reactive oxygen species scavenger: Importance of cysteine residues. J. Biol. Chem. 288(16), 11611-11620.
22. Poole, L. B. (2015) The basics of thiols and cysteines in redox biology and chemistry. Free Radic. Biol. Med. 80, 148-57
23. Conte, M. L. and Carroll, K. S. (2013) The redox biochemistry of protein sulfenylation and sulfinylation. J. Biol. Chem. 288(37), 26480-26488.
24. Cheung, H. S., Wang, S-B., Venkatraman, V., Murray, C. I. and Van Eyk., J. E. (2013) Cysteine oxidative posttranslational modifications. Emerging regulation in the cardiovascular system. Circ. Res. 112, 382-392
25. Wasil, M., Halliwell, B., Hutchison, D. C. S. and Baum, H. (1987) The antioxidant action of human extracellular fluids. Effect of human serum and its protein components on the inactivation of α₁-antiproteinase by hypochlorous acid and by hydrogen peroxide. Biochem. J. 243, 219-223.
26. Li, Z., Alam, S., Wang, J., Sandstrom, C. S., Janciauskiene, S. and Mahadeva, R. (2009) Oxidized α₁-antitrypsin stimulates the release of monocyte chemotactic protein-1 from lung epithelial cells: potential role in emphysema. Am. J. Physiol. Lung Cell. Mol. Physiol., 297(2), L388-400.
27. Mangan, M. S. J., Bird, C. H., Kaiserman, D., Matthews, A. Y., Hitchen, C., Steer, D. L., Thompson, P. E. and Bird, P. I. (2016) A Novel serpin regulatory mechanism. SerpinB9 is reversibly inhibited by vicinal disulphide bond formation in the reactive center loop. J. Biol. Chem. 291(7), 3626-3638.
28. Burgener, S. S., Leborgne, L. G. F., Snipas, S. J., Salvesen, G. S., Bird, P. I., and Benarafa, C. (2019) Cathepsin G inhibition by serpin b1 and serpin b6 prevents programmed necrosis in neutrophils and monocytes and reduces GSDMD-driven inflammation. Cell Reports. 27, 3646-3656.
29. Driessen, H. P., de Jong, W. W., Tesser, G. I., and Bloementhal, H. (1985) The mechanism of N-terminal acetylation of proteins. CRC Crit Rev Biochem. 18(4), 281-325.
30. Horvath, A. J., Lu, B. G., Pike, R. N., and Bottomly, S. P. (2011) Methods to measure the kinetics of protease inhibition by serpins. Methods Enzymol. 501, 223-235.
31. Taggart, C., Cervantes-Laurean, D., Kim, G., McElvaney, N. G., Wehr, N., Moss, J., and Levine R. L. (2000) Oxidation of either methionine 351 or methionine 358 in alpha 1-antitrypsin causes loss of anti-neutrophil elastase activity. J. Biol. Chem. 275(35), 27258-65
32. Church, D. F., and Pryor, W. A. (1985) Free-radical chemistry of cigarette smoke and its toxicological implications. Environ health Perspec. 64, 111-126
33. Sussan, T. E., Gajghate, S., Thimulappa, R. K., Ma. J., Kim, J-H., Sudini, K., Consolini, N., Cormier, S. A., Lomnicki, S., Hasan, F., Pekosz., A., and Biswal, S. (2015) Exposure to electronic cigarettes impairs pulmonary anti-bacterial and anti-viral defenses in a mouse model. PLOS ONE, 10(2), e0116861
34. Rock, K. L., Latz, E., Ontiveros, F., and Kono, H. (2010) The sterile inflammatory response. Annu Rev Immunol. 28, 321-342
35. Kirchner, T., Moller, S., Klinger, M., Solbach, W., Laskay, T., and Behnen, M. (2012) The impact of various reactive oxygen species on the formation of neutrophil extracellular traps. Mediators Inflamm. 2012, 849136.
36. Stoiber, W., Obermayer, A., Steinbacher, P., and Krautgartner, W-D., (2015) The role of reactive oxygen species (ROS) in the formation of extracellular traps (ETs) in humans. Biomolecules, 5, 702-723.
37. Bjornsdottir, H., Welin, A., Michaelson, E., Osla, V., Berg, S., Christenson, K., Sundqvist, M., Dahlgren, C., Karlsson, A., and Bylund, J. (2015) Neutrophil NET formation is regulated from the inside by myeloperoxidase-processed reactive oxygen species. Free Radic. Biol. Med. 89, 1024-35.
38. Wang, Z., Nicholls, S. J., Rodriguez, E. R., Kummu, O., Horkko, S., Barnard, J., Reynolds, W. F., Topol, E. J., DiDonato, J. A., and Hazen, S. L. (2007) Protein carbamylation links inflammation, smoking, uremia and atherogenesis. Nat Med. 13(10), 1176-84.
39. Pierterse, E., Rother, N., Yanginlar, C., Gerretsen, J., Boeltz, S., Munoz, L. E., Herrmann, M., Pickkers, P., Hilbrands, L. B., and van der Vlag, J. (2018) Cleaved N-terminal histone tails distinguish between NADPH oxidase (NOX)-dependent and NOX-independent pathways of neutrophil extracellular trap formation. Ann Rheum Dis. 77(12), 1790-98.
40. Hampton, M. B., Stamenkovic, I., and Winterbourn, C. C. (2002) Interaction with substrate sensitizes caspase-3 to inactivation by hydrogen peroxide. FEBS Lett. 517, 229-232.
41. Remijsen, Q., Kuijpers, T. W., Wirawan, E., Lippens, S., Vandenabeele, P., and VandenBerghe, T (2011). Dying for a cause: NETosis, mechanisms behind an antimicrobial cell death modality. Cell Death Differ. 18, 581-588.
42. Subramaniam, R., Dassanayake, R. P., Noromine, J., Brown, W. C., Knowles, D. P., and Srikumaran, S. (2010) Molecular cloning, characterization and in vitro expression of SERPIN B1 of bighorn sheep (Ovis Canadensis) and domestic sheep ((Ovis aries) and comparison with that of other species. Veterinary Immunology and Immunopathology. 137, 327-331.
43. Loison, F., Zhu, H., Karatepe, K., Kasorn, A., Liu, P., Ye, K., Zhou, J., Cao, S., Gong, H., Jenne, D. E., Remold-O'Donnell, E., Xu, Y., and Luo, H. R. (2014) Proteinase 3-dependent caspase-3 cleavage modulates neutrophil death and inflammation. J Clin. Invest. 124(10), 4445-4458.
44. Shi, J., Zhao, Y., Wang, K., Shi, X., Huang, H., Zhuang, Y., Cai, T., Wang, F., and Shao, F. (2015) cleavage of DSDMD by inflammatory caspases determines pyroptotic cell death. Nature. 526, 660-665.
45. Metzler, K. D., Goosman, C., Lubojemska, A., Zychlinsky, A., and Papayannopoulos, V. (2014) A myeloperoxidase-containing complex regulates neutrophil elastase release and actin dynamics during NETosis. Cell Reports. 8, 883-896.
46. Pappayannopoulos, V., Metzler, K. D., Hakim, A., and Zychlinsky, A. (2010) Neutrophil elastase and myeloperoxidase regulate the formation of neutrophil extracellular traps. J Cell Biol. 191, 677-691.

All publications and patent documents cited herein are incorporated herein by reference as if each such publication or document was specifically and individually indicated to be incorporated herein by reference. Although the present invention is described primarily with reference to specific embodiments, it is also envisioned that other embodiments will become apparent to those skilled in the art upon reading the present disclosure, and it is intended that such embodiments be contained within the present inventive methods.

Table of Illustrative sequences

```
SEQ ID NO: 1:
Protein Sequence of Human MNE1 (SERPIN B1)
wild type C344 (the underlined is
residue C344)
    1 meqlssantr faldlflals ennpagnifi
        spfsissama mvflgtrgnt aaqlsktfhf
  61 ntveevhsrf qslnadinkr gasyilklan
        rlygektynf lpeflvstqk tygadlasvd
121 fqhasedark tinqwvkgqt egkipellas
        gmvdnmtklv lvnaiyfkgn wkdkfmkeat
181 tnapfrlnkk drktvkmmyq kkkfaygyie
        dlkcrvlelp yqgeelsmvi llpddiedes
241 tglkkieeql tleklhewtk penldfievn
        vslprfklee sytlnsdlar lgvqdlfnss
301 kadlsgmsga rdifiskivh ksfvevneeg
        teaaaatagi atfcmlmpee nftadhpflf
361 firhnssgsi lflgrfssp
SEQ ID NO: 2:
Protein Sequence of Human MNEI
C344G variant (the underlined
shows the substitution at
residue C344)
    1 meqlssantr faldlflals ennpagnifi
        spfsissama mvflgtrgnt aaqlsktfhf
  61 ntveevhsrf qslnadinkr gasyilklan
        rlygektynf lpeflvstqk tygadlasvd
121 fqhasedark tinqwvkgqt egkipellas
        gmvdnmtklv lvnaiyfkgn wkdkfmkeat
181 tnapfrlnkk drktvkmmyq kkkfaygyie
        dlkcrvlelp yqgeelsmvi llpddiedes
241 tglkkieeql tleklhewtk penldfievn
        vslprfklee sytlnsdlar lgvqdlfnss
301 kadlsgmsga rdifiskivh ksfvevneeg
        teaaaatagi atfgmlmpee nftadhpflf
361 firhnssgsi lflgrfssp
SEQ ID NO: 3:
Protein Sequence of Human MNEI
C344A variant (the underlined
shows the substitution at
residue C344)
    1 meqlssantr faldlflals ennpagnifi
        spfsissama mvflgtrgnt aaqlsktfhf
  61 ntveevhsrf qslnadinkr gasyilklan
        rlygektynf lpeflvstqk tygadlasvd
121 fqhasedark tinqwvkgqt egkipellas
        gmvdnmtklv lvnaiyfkgn wkdkfmkeat
181 tnapfrlnkk drktvkmmyq kkkfaygyie
        dlkcrvlelp yqgeelsmvi llpddiedes
241 tglkkieeql tleklhewtk penldfievn
        vslprfklee sytlnsdlar lgvqdlfnss
301 kadlsgmsga rdifiskivh ksfvevneeg
        teaaaatagi atfamlmpee nftadhpflf
361 firhnssgsi lflgrfssp
SEQ ID NO: 4:
Protein Sequence of Human MNEI
C344V variant (the underlined
shows the substitution at
residue C344)
    1 meqlssantr faldlflals ennpagnifi
        spfsissama mvflgtrgnt aaqlsktfhf
  61 ntveevhsrf qslnadinkr gasyilklan
        rlygektynf lpeflvstqk tygadlasvd
121 fqhasedark tinqwvkgqt egkipellas
        gmvdnmtklv lvnaiyfkgn wkdkfmkeat
181 tnapfrlnkk drktvkmmyq kkkfaygyie
        dlkcrvlelp yqgeelsmvi llpddiedes
241 tglkkieeql tleklhewtk penldfievn
        vslprfklee sytlnsdlar lgvqdlfnss
301 kadlsgmsga rdifiskivh ksfvevneeg
        teaaaatagi atfvmlmpee nftadhpflf
361 firhnssgsi lflgrfssp
SEQ ID NO: 5:
Protein Sequence of Human MNEI
C344S variant (the underlined
shows the substitution at residue C344)
    1 meqlssantr faldlflals ennpagnifi
        spfsissama mvflgtrgnt aaqlsktfhf
  61 ntveevhsrf qslnadinkr gasyilklan
        rlygektynf lpeflvstqk tygadlasvd
121 fqhasedark tinqwvkgqt egkipellas
        gmvdnmtklv lvnaiyfkgn wkdkfmkeat
181 tnapfrlnkk drktvkmmyq kkkfaygyie
        dlkcrvlelp yqgeelsmvi llpddiedes
241 tglkkieeql tleklhewtk penldfievn
        vslprfklee sytlnsdlar lgvqdlfnss
301 kadlsgmsga rdifiskivh ksfvevneeg
        teaaaatagi atfsmlmpee nftadhpflf
361 firhnssgsi lflgrfssp
SEQ ID NO: 6
DNA Sequence of Yeast ADH2 promoter
(accession #JO1314 M13475 VO 1293
TATACCAATGGCAAACTGAGCACAACAATACCAGTC
CGGATCAACTGGCACCATCTCTCCCGTAGTCTCAT
CTAATTTTTCTTCCGGATGAGGTTCCAGATATACC
GCAACACCTTTATTATGGTTTCCCTGAGGGAATAA
TAGAATGTCCCATTCGAAATCACCAATTCTAAACC
TGGGCGAATTGTATTTCGGGTTTGTTAACTCGTTC
CAGTCAGGAATGTTCCACGTGAAGCTATCTTCCAG
CAAAGTCTCCACTTCTTCATCAAATTGTGGAGAAT
ACTCCCAATGCTCTTATCTATGGGACTTCCGGGAA
ACACAGTACCGATACTTCCCAATTCGTCTTCAGAG
CTCATTGTTTGTTTGAAGAGACTAATCAAAGAATC
GTTTTCTCAAAAAAATTAATATCTTAACTGATAGT
TTGATCAAAGGGGCAAAACGTAGGGGCAAACAAAC
GGAAAAATCGTTTCTCAAATTTTCTGATGCCAAGA
ACTCTAACCAGTCTTATCTAAAAATTGCCTTATGA
TCCGTCTCTCCGGTTACAGCCTGTGTAACTGATTA
ATCCTGCCTTTCTAATCACCATTCTAATGTTTTAA
TTAAGGGATTTTGTCTTCATTAACGGCTTTCGCTC
ATAAAAATGTTATGACGTTTTGCCCGCAGGCGGGA
AACCATCCACTTCACGAGACTGATCTCCTCTGCCG
GAACACCGGGCATCTCCAACTTATAAGTTGGAGAA
ATAAGAGAATTTCAGATTGAGAGAATGAAAAAAAA
AAAAAAAAAAAAGGCAGAGGAGAGCATAGAAATGG
GGTTCACTTTTTGGTAAAGCTATAGCATGCCTATC
ACATATAAATAGAGTGCCAGTAGCGACTTTTTTCA
CACTCGAAATACTCTTACTACTGCTCTCTTGTTGT
TTTTATCACTTCTTGTTTCTTCTTGGTAAATAGAA
TATCAAGCTACAAAAAGCATACAATCAACTATCAA
CTATTAACTATATCGTAAT
```

1. A SERPIN B1 variant polypeptide, wherein the SERPIN B1 variant polypeptide comprises an amino acid sequence that is at least 90% identical to SEQ ID NO: 1, and wherein the SERPIN B1 variant polypeptide possesses neutrophil or pancreatic elastase inhibitory activity and the neutrophil or pancreatic elastase inhibition activity is resistant to oxidation by free radicals.

2. The SERPIN B1 variant polypeptide of claim 1, wherein the free radicals are reactive oxygen species, reactive nitrogen species, or both.

3. The SERPIN B1 variant polypeptide of claim 1, wherein the SERPIN variant polypeptide comprises an amino acid substitution that is selected from the group consisting of C344A, C344V, and C344G, as compared to the sequence of SEQ ID NO: 1.

4. The SERPIN B1 variant polypeptide of claim 1, wherein the Serpin B1 variant polypeptide is fused to an Fc portion of an IgG or a single chain variable fragment (scFv) of an antibody, or wherein the SERPIN B1 variant polypeptide is pegylated.

5. (canceled)

6. A polynucleotide encoding the SERPIN B1 variant polypeptide of claim 1.

7. A pharmaceutical composition comprising the SERPIN variant polypeptide of claim 1 and a pharmaceutically acceptable excipient.

8. A pharmaceutical composition comprising a SERPIN B1 polypeptide comprising an amino acid sequence that is at least 90% identical to SEQ ID NO: 1 and a reducing agent that prevents oxidation of cysteine 344, wherein the polypeptide is capable of inhibiting a neutrophil or a pancreatic elastase.

9. The pharmaceutical composition of claim 8, wherein the reducing agent is N-acetylcysteine (NAC).

10. A method of treating a patient having a disease that is associated with increased production of free radicals as compared to a normal individual or increased exposure to free radicals in the environment,
wherein the method comprises administering the SERPIN B1 variant polypeptide of claim 1 to the patient, wherein the SERPIN B1 variant polypeptide comprises an amino acid substitution at residue 344, as compared to the native protein sequence of SEQ ID NO: 1;
wherein the SERPIN B1 variant polypeptide is capable of inhibiting the serine protease activity of neutrophil or pancreatic elastase; and
wherein the SERPIN B1 variant polypeptide is resistant to oxidation by a free radical.

11. The method of claim 10, wherein the free radical is a reactive oxygen species, a reactive nitrogen species, or both.

12. The method of claim 10, wherein the SERPIN B1 variant polypeptide comprises an amino acid sequence selected from the group consisting of SEQ ID NO: 2, SEQ ID NO: 3, and SEQ ID NO: 4.

13. (canceled)

14. A method of treating a patient having a disease that is associated with increased production of free radicals as compared to a normal individual or increased exposure to free radicals in the environment,
wherein the method comprises administering an effective amount of the pharmaceutical composition of claim 8 to the patient.

15. The method of claim 10, wherein the disease is associated with exposure to free radicals present in cigarette smoke or increased production of free radicals by enzymes present in innate immune cells, mucosal cells or glandular cells as compared to a normal individual.

16. The method of claim 10, wherein the disease is selected from the group consisting of an infectious disease, an autoimmune disease, a respiratory disease, a metabolic disease, a cardiovascular disease, a neurodegenerative, and an oncology disease.

17. The method of claim 10, wherein the the SERPIN B1 variant polypeptide is administered by inhalation, intra-tracheally, topically or by injection subcutaneously, intravenously, or intraperitoneally.

18. The method of claim 17, wherein the SERPIN B1 variant polypeptide is administered at a dose of 0.01 mg/kg to 1000 mg/kg.

19. A method of producing a native SERPIN B1 polypeptide or a SERPIN B1 variant polypeptide, the method comprising:
expressing a polynucleotide encoding the native SERPIN B1 polypeptide or the SERPIN B1 variant polypeptide in S. cerevisiae,
wherein the SERPIN B1 variant polypeptide comprises an amino acid sequence that is at least 90% identical to SEQ ID NO: 1, wherein the SERPIN B1 variant polypeptide comprises an amino acid substitution at residue 344, as compared to the native protein sequence of SEQ ID NO: 1,
wherein the SERPIN B1 variant polypeptide is capable of inhibiting the serine protease activity of neutrophil or pancreatic elastase, and
wherein the SERPIN B1 variant polypeptide is resistant to oxidation by free radicals.

20. The method of claim 19, wherein the S. cerevisiae is protease-deficient.

21. The method of claim 19, wherein the method step of expressing the polynucleotide is by introducing a Yeast episomal expression plasmid (YEp) into the S. cerevisiae.

22. The method of claim 19, wherein the polynucleotide is linked to a yeast promoter.

23. The method of claim 22, wherein the yeast promoter is an ADH 2 promoter.

24. The method of claim 19, wherein the polynucleotide is codon optimized for expression in yeast.

25. The method of claim 19, wherein the Serpin B1 variant polypeptide is fused to an Fc portion of an IgG or a single chain variable fragment (scFv) of an antibody, or wherein the Serpin B1 variant polypeptide is pegylated.
